(19) United States
(12) Patent Application Publication
(10) Pub. No.: US 2021/0095335 A1
(43) Pub. Date: Apr. 1, 2021

(54) METHOD FOR QUANTIFYING TARGET NUCLEIC ACIDS

(71) Applicant: Mitsubishi Chemical Corporation, Tokyo (JP)

(72) Inventors: Kazuki Momose, Tokyo (JP); Ai Hara, Tokyo (JP); Naoyuki Togawa, Tokyo (JP); Eiji Satou, Tokyo (JP)

(73) Assignee: Mitsubishi Chemical Corporation, Tokyo (JP)

(21) Appl. No.: 17/100,484

(22) Filed: Nov. 20, 2020

Related U.S. Application Data
(63) Continuation of application No. PCT/JP2019/020610, filed on May 24, 2019.

(30) Foreign Application Priority Data
May 25, 2018 (JP) 2018-100475
Feb. 18, 2019 (JP) 2019-026519

(51) Int. Cl. C12Q 1/6848; C12Q 1/6897; C12Q 1/6853; C12Q 1/6811; C12Q 1/686

(52) U.S. Cl. CPC C12Q 1/6848; C12Q 1/6897; C12Q 1/6853; C12Q 2545/10; C12Q 1/686; C12Q 2565/101; C12Q 1/6811

(57) ABSTRACT

A method for determining absolute amounts of two or more types of target nucleic acids in a sample, including: (a) mixing a sample with a known amount of a control nucleic acid; (b) co-amplifying all target nucleic acids and a specific nucleic acid group in the sample and the control nucleic acid; (c) determining a total amount of the all target nucleic acids and the specific nucleic acid group in the sample based on an indicator of a total amount of the amplified all target nucleic acids and the amplified specific nucleic acid group and an indicator of an amount of the amplified control nucleic acid; and (d) calculating an absolute amount of each target nucleic acid in the sample based on an occupancy of each target nucleic acid in the total of the all target nucleic acids and the specific nucleic acid group.

### TECHNICAL FIELD

The present invention relates to a method for quantifying nucleic acids and an analysis kit utilizing the same.

### BACKGROUND ART

There are many fields in need of a simultaneous quantification of multiple types of nucleic acids in a sample and one example is bacterial flora analysis.

In recent years, the relationship between human health and oral bacterial florae and intestinal bacterial florae has become evident thereby demanding techniques for analyzing bacterial florae in a simple manner for the purpose of examinations.

Microscopy and culture method have been traditionally used as tools to evaluate bacterial florae but pose a problem of limiting bacterial species applicable to be discriminated and quantified. Instead of these methods, molecular biological techniques based on the specificity of a nucleic acid sequence has been used in recent years. Quantitative PCR (qPCR), competitive PCR, DNA chips, next generation sequencer (NGS) are known as such a molecular biological technique.

qPCR method commonly used enables highly sensitive quantification but requires cumbersome operations and has a limited number of items applicable to examinations as in competitive PCR. DNA chip is a high-throughput screening but the quantification using this is a relative quantification. NGS requires cumbersome operations and is expensive thereby involving challenges to be used for the purpose of routine diagnosis.

At moment, bacterial flora analysis using a DNA chip is incapable of carrying out absolute quantification. When bacterial flora analysis is carried out using a DNA chip or NGS, a nucleic acid group comprising target nucleic acids needs to be amplified by a technique such as PCR method. The measurement result obtained by the technique represents a relative relationship between a plurality of targets by the amplification.

There is competitive PCR as the technique for determining an absolute amount of a nucleic acid in a simple manner. Competitive PCR is a method in which a control nucleic acid in a known concentration and to be co-amplified with a target is added before amplification for determining an absolute amount before amplification from an indicator of an amount after amplification. However, in this competitive PCR, only one type of a nucleic acid can be an object for quantification and it is difficult to quantify a plural type of nucleic acids simultaneously.

Additionally, a technique for bacterial flora analysis in which a control nucleic acid in a known concentration is added before amplification to study an existing amount thereof is already known (Patent Literature 1, Non Patent Literature 1). For example, in Non Patent Literature 1, a control nucleic acid is added and relative amount ratios among samples are compared after PCR amplification using a DNA chip. In a DNA chip, every probe has different hybridization efficiency thereby failing to compare signals among different probes and thus this method cannot determine an absolute amount.

On the other hand, Patent Literature 1 discloses, for the purpose of absolute quantification by NGS, a method in which a plurality of control nucleic acids are added before amplification and a calibration curve is created using only such a plurality of control nucleic acids thereby to determine an amount of each target nucleic acid. This method has a premise that the amplification efficiency of the control nucleic acids and target nucleic acids is identical and Patent Literature 1 proposes sequences artificially designed so that the amplification efficiency becomes identical. This method is a cumbersome method which uses an expensive NGS and further a plurality of control nucleic acids. Additionally, this method involves an extremely difficult problem in that the amplification efficiency between a target nucleic acid and a plurality of control nucleic acids in a sample must be identical.

As described above, a method capable of simultaneously quantifying absolute amounts of a plural type of target nucleic acids in a simple manner has not been known.

### CITATION LIST

### Patent Literature

[Patent Literature 1] Japanese Patent Laid-Open No. 2015-204813

### Non Patent Literature

[Non Patent Literature 1] Treimo, J. et al. J Appl Microbiol, 100, 985-998, 2006

### SUMMARY OF INVENTION

### Technical Problem

Thus, the present invention has an object to provide a simple means capable of simultaneously determining absolute amounts of two or more types of target nucleic acids in a sample.

### Solution to Problem

The present inventors conducted extensive studies to solve the above problems and consequently found that the step of calculating an absolute quantification of the total of all target nucleic acids and a specific nucleic acid group to be collectively amplified with the target nucleic acids in a sample using a control nucleic acid and a breakdown thereof enables the absolute quantification of two or more types of target nucleic acids respectively in the sample, whereby the present invention has been accomplished.

That is, the present invention is as follows.

[1] A method for determining absolute amounts of two or more types of target nucleic acids in a sample, the method comprising the steps of:

(a) mixing a sample with a known amount of a control nucleic acid;

(b) co-amplifying all target nucleic acids and a specific nucleic acid group to be collectively amplified with the target nucleic acids in the sample and the control nucleic acid;

(c) determining a total amount of all target nucleic acids and the specific nucleic acid group in the sample based on an indicator of a total amount of the amplified all target nucleic acids and the specific nucleic acid group and an indicator of an amount of the amplified control nucleic acid; and

(d) calculating an absolute amount of each target nucleic acid in the sample based on an occupancy of each target nucleic acid in the total of all target nucleic acids and the specific nucleic acid group which is calculated from the indicator of the total amount of the amplified all target nucleic acids and the specific nucleic acid group and an indicator of an amount of each amplified target nucleic acid.

[2] The method according to [1], wherein the control nucleic acid is an artificial sequence consisting of a base sequence required for amplification and a base sequence in which a part or all of the bases are in a random combination.
[3] The method according to [1] or [2], wherein the co-amplification of the total of all target nucleic acids and the specific nucleic acid group in the sample and the control nucleic acid is carried out by a technique selected from the group consisting of Polymerase Chain Reaction (PCR) method, Loop-Mediated Isothermal Amplification (LAMP) method, and in vitro transcription.
[4] The method according to [3], wherein the co-amplification of the total of all target nucleic acids and the specific nucleic acid group in the sample and the control nucleic acid is carried out by PCR method.
[5] The method according to any of [1] to [4], wherein the sample is at least one selected from the group consisting of foods and drinks, biological samples, environmental samples, and samples from industrial process.
[6] The method according to [5], wherein the sample is at least one selected from the group consisting of activated sludges, soils, river waters, seawaters, hot spring waters, drinking waters, processed foods, fermenter cultures, and tissues, cells and body fluids collected from eukaryotes.
[7] The method according to [5], wherein the biological sample is at least one selected from the group consisting of saliva, plaque, gingival crevicular fluid (GCF), feces, and skin-derived samples collected from mammals.
[8] The method according to any of [1] to [7], wherein the target nucleic acid is a bacterial ribosomal RNA (rRNA) gene.
[9] The method according to [8], wherein the target nucleic acids are rRNA genes of at least two species of bacteria selected from the group consisting of bacteria belonging to the Abiotrophia genus, Achromobacter genus, Acinetobacter genus, Actinomyces genus, Aerococcus genus, Aggregatibacter genus, Alloprevotella genus, Alloscardovia genus, Anaerococcus genus, Anaeroglobus genus, Arcanobacterium genus, Atopobium genus, Bacillus genus, Bacteroides genus, Bifidobacterium genus, Bordetella genus, Brevundimonas genus, Brucella genus, Burkholderia genus, Campylobacter genus, Candidatus Absconditabacteria (SR1), Candidatus Saccharibacteria (TM7), Capnocytophaga genus, Cardiobacterium genus, Catonella genus, Chlamydia genus, Chlamydophila genus, Chryseobacterium genus, Citrobacter genus, Clostridium genus, Collinsella genus, Corynebacterium genus, Coxiella genus, Cronobacter genus, Cryptobacterium genus, Curvibacter genus, Dialister genus, Eggerthella genus, Eikenella genus, Elizabethkingia, Enterobacter genus, Enterococcus genus, Escherichia genus, Eubacterium genus, Filifactor genus, Finegoldia genus, Fusobacterium genus, Gardnerella genus, Gemella genus, Granulicatella genus, Haemophilus genus, Helicobacter genus, Kingella genus, Klebsiella genus, Kocuria genus, Lachnoanaerobaculum genus, Lactobacillus genus, Lactococcus genus, Lautropia genus, Legionella genus, Leptotrichia genus, Listeria genus, Megasphaera genus, Methanobrevibacter genus, Microbacterium genus, Micrococcus genus, Mitsuokella genus, Mobiluncus genus, Mogibacterium genus, Moraxella genus, Morganella genus, Mycobacterium genus, Mycoplasma genus, Neisseria genus, Nocardia genus, Olsenella genus, Oribacterium genus, Paracoccus genus, Parascardovia genus, Parvimonas genus, Peptoniphilus genus, Peptostreptococcus genus, Porphyromonas genus, Prevotella genus, Propionibacterium (Cutibacterium) genus, Proteus genus, Providencia genus, Pseudomonas genus, Pseudopropionibacterium genus, Pseudoramibacter genus, Pyramidobacter genus, Ralstonia genus, Rothia genus, Scardovia genus, Schlegelella genus, Sebaldella genus, Selenomonas genus, Serratia genus, Simonsiella genus, Slackia genus, Sneathia genus, Solobacterium genus, Staphylococcus genus, Stenotrophomonas genus, Stomatobaculum genus, Streptococcus genus, Tannerella genus, Treponema genus, Ureaplasma genus, Veillonella genus, and Fretibacterium genus.
[10] The method according to [8], wherein the target nucleic acids are rRNA genes of at least two species of bacteria selected from the group consisting of Abiotrophia defectiva, Achromobacter xylosoxidans, Acinetobacter baumannii, Acinetobacter calcoaceticus, Acinetobacter johnsonii, Acinetobacter junii, Acinetobacter lwoffii, Actinomyces dentalis, Actinomyces georgiae, Actinomyces gerencseriae, Actinomyces graevenitzii, Actinomyces israelii, Actinomyces johnsonii, Actinomyces meyeri, Actinomyces naeslundii, Actinomyces odontolyticus, Actinomyces oris, Actinomyces turicensis, Actinomyces viscosus, Aggregatibacter actinomycetemcomitans, Aggregatibacter aphrophilus, Alloprevotella rava, Alloprevotella tannerae, Alloscardovia omnicolens, Anaeroglobus geminatus, Atopobium parvulum, Bacillus cereus, Bacteroides fragilis, Bifidobacterium adolescentis, Bifidobacterium bifidum, Bifidobacterium breve, Bifidobacterium dentium, Bifidobacterium longum, Bordetella pertussis, Brucella abortus, Burkholderia cepacia, Campylobacter concisus, Campylobacter gracilis, Campylobacter rectus, Campylobacter showae, Capnocytophaga gingivalis, Capnocytophaga ochracea, Capnocytophaga sputigena, Cardiobacterium hominis, Catonella morbi, Chlamydia psittaci, Chlamydia trachomatis, Chlamydia pneumoniae, Chryseobacterium indologenes, Citrobacter freundii, Clostridium perfringens, Collinsella aerofaciens, Corynebacterium diphtheriae, Corynebacterium durum, Corynebacterium matruchotii, Corynebacterium pseudodiphtheriticum, Corynebacterium pseudotuberculosis, Corynebacterium renale, Corynebacterium striatum, Corynebacterium xerosis, Coxiella burnetii, Cryptobacterium curtum, Curvibacter delicatus, Dialister invisus, Dialister micraerophilus, Dialister pneumosintes, Eggerthella lenta, Eikenella corrodens, Elizabethkingia meningoseptica, Enterobacter aerogenes, Enterobacter cloacae, Enterococcus avium, Enterococcus faecalis, Enterococcus faecium, Escherichia coli, Eubacterium brachy, Eubacterium infirmum, Eubacterium limosum, Eubacterium minutum, Eubacterium nodatum, Eubacterium saphenum, Eubacterium sulci, Filifactor alocis, Fretibacterium fastidiosum, Fusobacterium necrophorum, Fusobacterium nucleatum, Fusobacterium nucleatum subsp. animalis, Fusobacterium nucleatum subsp. nucleatum, Fusobacterium nucleatum subsp. polymorphum, Fusobacterium nucleatum subsp. vincentii, Fusobacterium periodonticum, Fusobacterium simiae, Gemella haemolysans, Gemella morbillorum, Gemella sanguinis, Granulicatella adiacens, Granulicatella balaenopterae, Granulicatella elegans, Haemophilus ducreyi, Haemophilus haemolyticus, Haemophilus influenzae, Haemophilus parainfluenzae, Helicobacter pylori, Kingella denitrificans, Kingella kingae, Kingella oralis, Klebsiella oxytoca, Klebsiella pneumoniae, Lachnoanaerobaculum orale, Lachnoanaerobaculum saburreum, Lactobacillus acidophilus, Lactobacillus brevis, Lactobacillus buchneri, Lactobacillus casei, Lactobacillus crispatus, Lactobacillus delbrueckii, Lactobacillus fermentum, Lactobacillus gasseri, Lactobacillus paracasei, Lactobacillus plantarum, Lactobacillus reuteri, Lactobacillus rhamnosus, Lactobacillus salivarius, Lautropia mirabilis, Legionella pneumophila, Leptotrichia buccalis, Leptotrichia sp. OT 215, Leptotrichia sp. OT 417, Leptotrichia sp. OT 462, Listeria monocytogenes, Megasphaera micronuciformis, Micrococcus luteus, Mogibacterium diversum, Mogibacterium neglectum, Mogibacterium pumilum, Mogibacterium timidum, Mogibacterium vescum, Moraxella catarrhalis, Moraxella lacunata, Morganella morganii, Mycobacterium abscessus, Mycobacterium avium, Mycobacterium chelonae, Mycobacterium fortuitum, Mycobacterium gordonae, Mycobacterium haemophilum, Mycobacterium intracellulare, Mycobacterium kansasii, Mycobacterium marinum, Mycobacterium szulgai, Mycobacterium tuberculosis, Mycobacterium xenopi, Mycoplasma genitalium, Mycoplasma hominis, Mycoplasma orale, Mycoplasma pneumoniae, Mycoplasma salivarium, Neisseria elongata, Neisseria flava, Neisseria flavescens, Neisseria gonorrhoeae, Neisseria meningitidis, Neisseria mucosa, Neisseria sicca, Neisseria subflava, Nocardia asteroides, Nocardia brasilliensis, Nocardia farcinica, Nocardia nova, Olsenella uli, Oribacterium asaccharolyticum, Oribacterium parvum, Parascardovia denticolens, Parvimonas micra, Peptostreptococcus stomatis, Porphyromonas catoniae, Porphyromonas endodontalis, Porphyromonas gingivalis, Porphyromonas pasteri, Prevotella dentalis, Prevotella denticola, Prevotella histicola, Prevotella intermedia, Prevotella loescheii, Prevotella melaninogenica, Prevotella nigrescens, Prevotella oralis, Prevotella oris, Prevotella pallens, Prevotella salivae, Prevotella shahii, Prevotella veroralis, Propionibacterium acnes (Cutibacterium acnes), Propionibacterium granulosum (Cutibacterium granulosum), Propionibacterium propionicus (Pseudopropionibacterium propionicum), Proteus mirabilis, Proteus vulgaris, Providencia stuartii, Pseudomonas aeruginosa, Pseudomonas fluorescens, Pseudomonas putida, Pseudoramibacter alactolyticus, Ralstonia pickettii, Rothia aeria, Rothia dentocariosa, Rothia mucilaginosa, Scardovia inopinata, Schlegelella aquatica, Sebaldella termitidis, Selenomonas flueggei, Selenomonas noxia, Selenomonas sp. OT 478, Selenomonas sputigena, Serratia marcescens, Simonsiella muelleri, Slackia exigua, Solobacterium moorei, SR1 sp. OT 345, Staphylococcus aureus, Staphylococcus epidermidis, Staphylococcus saccharolyticus, Stenotrophomonas maltophilia, Streptococcus agalactiae, Streptococcus anginosus, Streptococcus australis, Streptococcus constellatus, Streptococcus cristatus, Streptococcus dentisani, Streptococcus gordonii, Streptococcus infantis, Streptococcus intermedius, Streptococcus milleri, Streptococcus mitis, Streptococcus mutans, Streptococcus oralis, Streptococcus parasanguinis, Streptococcus pneumoniae, Streptococcus pyogenes, Streptococcus salivarius, Streptococcus sanguinis, Streptococcus sobrinus, Streptococcus tigurinus, Streptococcus vestibularis, Tannerella forsythia, Treponema denticola, Treponema lecithinolyticum, Treponema medium, Treponema pallidum, Treponema socranskii, Treponema vincentii, Ureaplasma urealyticum, Veillonella atypica, Veillonella dispar, Veillonella parvula, and Veillonella rogosae.
[11] The method according to any of [1] to [10], wherein Step (c) comprises the steps of:

(c1) creating a calibration curve of relationships, using a part or all of the target nucleic acids and the specific nucleic acid group and the control nucleic acid, between amounts before amplification and indicators of amounts after amplification; and

(c2) calculating a total amount of all target nucleic acids and the specific nucleic acid group in the sample by applying an indicator of a total amount of the amplified all target nucleic acids and the specific nucleic acid group from the sample and an indicator of an amount of the amplified control nucleic acid to the calibration curve created in Step (c1).

[12] The method according to [11], wherein the nucleic acid used in Step (el) is a part of the target nucleic acids and the specific nucleic acid group.
[13] The method according to [11] or [12], wherein the calibration curve represents a relationship between

a ratio of an indicator of a total amount of a part or all of the target nucleic acids and the specific nucleic acid group to an indicator of an amount of the control nucleic acid after amplification and

a ratio of a total amount of a part or all of the target nucleic acids and the specific nucleic acid group to an amount of the control nucleic acid before amplification.

[14] The method according to any of [1] to [13], wherein, in Steps (c) and (d), an indicator of an amount of each amplified target nucleic acid, an indicator of a total amount of amplified all target nucleic acids and the specific nucleic acid group, and an indicator of an amount of the amplified control nucleic acid are signal intensities obtained by hybridization with the following probes (x) to (z), respectively, loaded on a DNA chip:

(x) a probe hybridizable respectively with amplified products of two or more types of target nucleic acids;

(y) a probe hybridizable in common with both amplified products of all target nucleic acids and the specific nucleic acid group in the sample; and

(z) a probe hybridizable with amplified products of the control nucleic acid.

[15] The method according to [14], wherein Step (d) comprises the steps of:

(d1) hybridizing respective target nucleic acids (each target nucleic acid) of all target nucleic acids in the sample with the DNA chip and calculating a relational expression on signal intensities between the probe (x) and the probe (y) hybridizable with each target nucleic acid,

(d2) converting the signal intensity of the probe (x) obtained by hybridizing products amplified from the sample with the DNA chip to a value equivalent to the signal intensity of the probe (y) hybridizable with each target nucleic acid using the relational expression calculated in Step (d1),

(d3) calculating a ratio of the value equivalent to the signal intensity of the probe (y) calculated in Step (d2) to the signal intensity of the probe (y) obtained by hybridizing products amplified from the sample with the DNA chip; and

(d4) calculating an absolute amount of each target nucleic acid in the sample by multiplying the total amount of all target nucleic acids and the specific nucleic acid group in the sample calculated in Step (c) by the occupancy calculated in Step (d3).

[16] The method according to [14] or [15], wherein Step (b) comprises the step of enriching nucleic acids after amplification for a single chain hybridizable with probes loaded on the DNA chip.
[17] The method according to [16], wherein the step of enriching for a single chain is carried out by asymmetric PCR or A, exonuclease treatment.
[18] The method according to any of [14] to [17], wherein the probe (x) is any of the following sequences:

(I) a sequence selected from the group consisting of the base sequences as set forth in SEQ ID NO: 3 to 191;

(II) a complementary sequence to (I); or (III) a sequence substantially identical with the sequence (I) or (II).

[19] The method according to any of [14] to [18], wherein the DNA chip is a fiber-type DNA chip.
[20] A kit comprising: one or two pairs of primer sets for co-amplifying all target nucleic acids and the specific nucleic acid group in a sample and a control nucleic acid by PCR method; a control nucleic acid; and a DNA chip on which the following probes (x) to (z) are loaded:

(x) a probe hybridizable respectively with amplified products of two or more types of target nucleic acids;

(y) a probe hybridizable in common with both amplified products of all target nucleic acids and the specific nucleic acid group in the sample; and

(z) a probe hybridizable with amplified products of the control nucleic acid.

[21] The kit according to [20], wherein the probe (x) is any of the following sequences:

(I) a sequence selected from the group consisting of the base sequences as set forth in SEQ ID NO: 3 to 191;

(II) a complementary sequence to (I); or

(III) a sequence substantially identical with the sequence (I) or (II).

[22] A DNA chip on which the following probes (x) to (z) are loaded:

(x) a probe hybridizable respectively with amplified products of two or more types of target nucleic acids;

(y) a probe hybridizable in common with both amplified products of all target nucleic acids and the specific nucleic acid group in the sample; and

(z) a probe hybridizable with amplified products of the control nucleic acid, wherein the probe (x) is any of the following sequences:

(IV) a sequence selected from the group consisting of the base sequences as set forth in SEQ ID NO: 43 to 191;

(V) a complementary sequence to (IV); or

(VI) a sequence substantially identical with the sequence (IV) or (V).

### Advantageous Effects of Invention

According to the present invention, absolute amounts of two or more types of target nucleic acids in a sample can be simultaneously determined in a simple manner.

### BRIEF DESCRIPTION OF DRAWINGS

FIG. 1 is a drawing showing the relationship on an amount ratio before amplification and a ratio of indicators of amounts after amplification between nucleic acids for calibration and a control nucleic acid.

FIG. 2 is drawings showing relationships on signal intensities among probes.

FIG. 3 is a drawing showing the relationship on an amount ratio before amplification and a ratio of indicators of amounts after amplification between nucleic acids for calibration and a control nucleic acid.

FIG. 4A is a schematic drawing showing a principle in reference to an example of the embodiments of the present invention and a drawing illustrating Step (a).

FIG. 4B is a schematic drawing showing a principle in reference to an example of the embodiments of the present invention and a drawing illustrating Step (b).

FIG. 4C is a schematic drawing showing a principle in reference to an example of the embodiments of the present invention and a drawing illustrating Step (c).

FIG. 4D is a schematic drawing showing a principle in reference to an example of the embodiments of the present invention and a drawing illustrating Step (d).

FIG. 5 is a drawing showing the relationship on an amount ratio before amplification and a ratio of indicators of amounts after amplification between nucleic acids for calibration and a control nucleic acid.

FIG. 6 is drawings showing relationships on signal intensities among probes.

FIG. 7 is a drawing showing the relationship on an amount ratio before amplification and a ratio of indicators of amounts after amplification between nucleic acids for calibration and a control nucleic acid.

FIG. 8 is drawings showing relationships on signal intensities among probes.

FIG. 9 is a drawing showing the relationship on an amount ratio before amplification and a ratio of indicators of amounts after amplification between nucleic acids for calibration and a control nucleic acid.

FIG. 10 is drawings showing relationships on signal intensities among probes.

### DESCRIPTION OF EMBODIMENTS

Hereinafter, the present invention will be described in detail. The following embodiments are examples to describe the present invention and do not intend to limit the present invention only to these embodiments. The present invention can be carried out in various modifications without departing from the spirit of the present invention. The present description incorporates the contents described in the specification and drawings of a Japanese patent application (Japanese Patent Application No. 2018-100475) filed on May 25, 2018 and a Japanese patent application (Japanese Patent Application No. 2019-026519) filed on Feb. 18, 2019, which serve as a basis of the priority of the present application.

### 1. Determination of Absolute Amounts of Target Nucleic Acids

### (1) Target Nucleic Acid

The target nucleic acid in the present invention is a nucleic acid to be quantified. For example, in a sample containing a bacterial genome DNA to be measured, a bacterial genome DNA not to be measured and other DNA, the target nucleic acids are the nucleic acids from bacteria to be measured. In the present invention, even if when a part of the nucleic acids from bacteria not to be measured is collectively amplified with the target nucleic acids, such a part of the amplified nucleic acids is not the target nucleic acid. Such a “the specific nucleic acid group to be collectively amplified with the target nucleic acids” will be described later.

For evaluating bacterial florae, as the target nucleic acids, nucleic acids comprising a consensus sequence conserved among genome DNA of a plurality of bacterial species to be measured can be used. Further, as the target nucleic acids, nucleic acids comprising a base sequence specific to respective bacteria to be measured can be used.

When the target nucleic acids comprise consensus sequences of a plurality of bacterial species, target nucleic acids (all target nucleic acids) of a plurality of bacterial species can be captured using a single type of probe. When the target nucleic acids comprise base sequences specific to respective bacteria to be measured, bacterial target nucleic acids (each target nucleic acid) of interest can be specifically detected.

A target nucleic acid may be one molecule of a nucleic acid or a plurality thereof may be present as a partial sequence in one molecule of a nucleic acid. For example, a bacterial 16SrRNA gene (for example, DNA encoding 16SrRNA) may be present in plurality copies in one molecule of a genome DNA but when these copies are considered as target nucleic acids, each copy can be considered as one molecule of the target nucleic acid, respectively. A nucleic acid may be either DNA or RNA.

In the present invention, the target nucleic acid further includes a nucleic acid mass which can be considered as a single type when obtaining an indicator of an amount of target nucleic acids. Specifically, a mass of nucleic acids comprising identical base sequence regions can be considered as a single type. For example, when a DNA chip is used, for a mass of nucleic acids having a partially common sequence, an indicator of an amount as a signal intensity of a probe hybridizable with such a common sequence can be obtained, and thereby the mass of nucleic acids can be considered as one target nucleic acid. Examples of this specifically include a case when the 16SrRNA genes of bacteria belonging to the Streptococcus genus in a sample are considered as target nucleic acids and an indicator of an amount is obtained from a signal intensity of a probe corresponding to the common sequence thereof.

Examples of the target nucleic acid include a genome DNA, a gene of interest in a genome DNA, and mRNA. More specifically, a bacterial genome DNA is preferable, a rRNA gene in a bacterial genome DNA is more preferable, and the 16SrRNA gene in a bacterial genome DNA is particularly preferable.

For evaluating oral bacterial florae, for example, rRNA genes of bacteria belonging to the Abiotrophia genus, Achromobacter genus, Acinetobacter genus, Actinomyces genus, Aerococcus genus, Aggregatibacter genus, Alloprevotella genus, Alloscardovia genus, Anaerococcus genus, Anaeroglobus genus, Arcanobacterium genus, Atopobium genus, Bacillus genus, Bacteroides genus, Bifidobacterium genus, Bordetella genus, Brevundimonas genus, Brucella genus, Burkholderia genus, Campylobacter genus, Candidatus Absconditabacteria (SR1), Candidatus Saccharibacteria (TM7), Capnocytophaga genus, Cardiobacterium genus, Catonella genus, Chlamydia genus, Chlamydophila genus, Chryseobacterium genus, Citrobacter genus, Clostridium genus, Collinsella genus, Corynebacterium genus, Coxiella genus, Cronobacter genus, Cryptobacterium genus, Curvibacter genus, Dialister genus, Eggerthella genus, Eikenella genus, Elizabethkingia, Enterobacter genus, Enterococcus genus, Escherichia genus, Eubacterium genus, Filifactor genus, Finegoldia genus, Fusobacterium genus, Gardnerella genus, Gemella genus, Granulicatella genus, Haemophilus genus, Helicobacter genus, Kingella genus, Klebsiella genus, Kocuria genus, Lachnoanaerobaculum genus, Lactobacillus genus, Lactococcus genus, Lautropia genus, Legionella genus, Leptotrichia genus, Listeria genus, Megasphaera genus, Methanobrevibacter genus, Microbacterium genus, Micrococcus genus, Mitsuokella genus, Mobiluncus genus, Mogibacterium genus, Moraxella genus, Morganella genus, Mycobacterium genus, Mycoplasma genus, Neisseria genus, Nocardia genus, Olsenella genus, Oribacterium genus, Paracoccus genus, Parascardovia genus, Parvimonas genus, Peptoniphilus genus, Peptostreptococcus genus, Porphyromonas genus, Prevotella genus, Propionibacterium (Cutibacterium) genus, Proteus genus, Providencia genus, Pseudomonas genus, Pseudopropionibacterium genus, Pseudoramibacter genus, Pyramidobacter genus, Ralstonia genus, Rothia genus, Scardovia genus, Schlegelella genus, Sebaldella genus, Selenomonas genus, Serratia genus, Simonsiella genus, Slackia genus, Sneathia genus, Solobacterium genus, Staphylococcus genus, Stenotrophomonas genus, Stomatobaculum genus, Streptococcus genus, Tannerella genus, Treponema genus, Ureaplasma genus, Veillonella genus and Fretibacterium genus can be considered as target nucleic acids, and more preferably rRNA genes of bacteria belonging to the Actinomyces genus, Aggregatibacter genus, Alloprevotella genus, Campylobacter genus, Candidatus Absconditabacteria (SR1), Capnocytophaga genus, Corynebacterium genus, Eikenella genus, Eubacterium genus, Filifactor genus, Fusobacterium genus, Gemella genus, Granulicatella genus, Haemophilus genus, Helicobacter genus, Lactobacillus genus, Leptotrichia genus, Megasphaera genus, Neisseria genus, Parvimonas genus, Peptostreptococcus genus, Porphyromonas genus, Prevotella genus, Rothia genus, Selenomonas genus, Solobacterium genus, Streptococcus genus, Tannerella genus, Treponema genus and Veillonella genus can be considered as target nucleic acids but are not limited thereto.

Further, for the target nucleic acid, rRNA genes of bacteria such as Abiotrophia defectiva, Achromobacter xylosoxidans, Acinetobacter baumannii, Acinetobacter calcoaceticus, Acinetobacter johnsonii, Acinetobacter junii, Acinetobacter lwoffii, Actinomyces dentalis, Actinomyces georgiae, Actinomyces gerencseriae, Actinomyces graevenitzii, Actinomyces israelii, Actinomyces johnsonii, Actinomyces meyeri, Actinomyces naeslundii, Actinomyces odontolyticus, Actinomyces oris, Actinomyces turicensis, Actinomyces viscosus, Aggregatibacter actinomycetemcomitans, Aggregatibacter aphrophilus, Alloprevotella rava, Alloprevotella tannerae, Alloscardovia omnicolens, Anaeroglobus geminatus, Atopobium parvulum, Bacillus cereus, Bacteroides fragilis, Bifidobacterium adolescentis, Bifidobacterium bifidum, Bifidobacterium breve, Bifidobacterium dentium, Bifidobacterium longum, Bordetella pertussis, Brucella abortus, Burkholderia cepacia, Campylobacter concisus, Campylobacter gracilis, Campylobacter rectus, Campylobacter showae, Capnocytophaga gingivalis, Capnocytophaga ochracea, Capnocytophaga sputigena, Cardiobacterium hominis, Catonella morbi, Chlamydia psittaci, Chlamydia trachomatis, Chlamydia pneumoniae, Chryseobacterium indologenes, Citrobacter freundii, Clostridium perfringens, Collinsella aerofaciens, Corynebacterium diphtheriae, Corynebacterium durum, Corynebacterium matruchotii, Corynebacterium pseudodiphtheriticum, Corynebacterium pseudotuberculosis, Corynebacterium renale, Corynebacterium striatum, Corynebacterium xerosis, Coxiella burnetii, Cryptobacterium curtum, Curvibacter delicatus, Dialister invisus, Dialister micraerophilus, Dialister pneumosintes, Eggerthella lenta, Eikenella corrodens, Elizabethkingia meningoseptica, Enterobacter aerogenes, Enterobacter cloacae, Enterococcus avium, Enterococcus faecalis, Enterococcus faecium, Escherichia coli, Eubacterium brachy, Eubacterium infirmum, Eubacterium limosum, Eubacterium minutum, Eubacterium nodatum, Eubacterium saphenum, Eubacterium sulci, Filifactor alocis, Fretibacterium fastidiosum, Fusobacterium necrophorum, Fusobacterium nucleatum, Fusobacterium nucleatum subsp. animalis, Fusobacterium nucleatum subsp. nucleatum, Fusobacterium nucleatum subsp. polymorphum, Fusobacterium nucleatum subsp. vincentii, Fusobacterium periodonticum, Fusobacterium simiae, Gemella haemolysans, Gemella morbillorum, Gemella sanguinis, Granulicatella adiacens, Granulicatella balaenopterae, Granulicatella elegans, Haemophilus ducreyi, Haemophilus haemolyticus, Haemophilus influenzae, Haemophilus parainfluenzae, Helicobacter pylori, Kingella denitrificans, Kingella kingae, Kingella oralis, Klebsiella oxytoca, Klebsiella pneumoniae, Lachnoanaerobaculum orale, Lachnoanaerobaculum saburreum, Lactobacillus acidophilus, Lactobacillus brevis, Lactobacillus buchneri, Lactobacillus casei, Lactobacillus crispatus, Lactobacillus delbrueckii, Lactobacillus fermentum, Lactobacillus gasseri, Lactobacillus paracasei, Lactobacillus plantarum, Lactobacillus reuteri, Lactobacillus rhamnosus, Lactobacillus salivarius, Lautropia mirabilis, Legionella pneumophila, Leptotrichia buccalis, Leptotrichia sp. OT 215, Leptotrichia sp. OT 417, Leptotrichia sp. OT 462, Listeria monocytogenes, Megasphaera micronuciformis, Micrococcus luteus, Mogibacterium diversum, Mogibacterium neglectum, Mogibacterium pumilum, Mogibacterium timidum, Mogibacterium vescum, Moraxella catarrhalis, Moraxella lacunata, Morganella morganii, Mycobacterium abscessus, Mycobacterium avium, Mycobacterium chelonae, Mycobacterium fortuitum, Mycobacterium gordonae, Mycobacterium haemophilum, Mycobacterium intracellulare, Mycobacterium kansasii, Mycobacterium marinum, Mycobacterium szulgai, Mycobacterium tuberculosis, Mycobacterium xenopi, Mycoplasma genitalium, Mycoplasma hominis, Mycoplasma orale, Mycoplasma pneumoniae, Mycoplasma salivarium, Neisseria elongata, Neisseria flava, Neisseria flavescens, Neisseria gonorrhoeae, Neisseria meningitidis, Neisseria mucosa, Neisseria sicca, Neisseria subflava, Nocardia asteroides, Nocardia brasilliensis, Nocardia farcinica, Nocardia nova, Olsenella uli, Oribacterium asaccharolyticum, Oribacterium parvum, Parascardovia denticolens, Parvimonas micra, Peptostreptococcus stomatis, Porphyromonas catoniae, Porphyromonas endodontalis, Porphyromonas gingivalis, Porphyromonas pasteri, Prevotella dentalis, Prevotella denticola, Prevotella histicola, Prevotella intermedia, Prevotella loescheii, Prevotella melaninogenica, Prevotella nigrescens, Prevotella oralis, Prevotella oris, Prevotella pallens, Prevotella salivae, Prevotella shahii, Prevotella veroralis, Propionibacterium acnes (Cutibacterium acnes), Propionibacterium granulosum (Cutibacterium granulosum), Propionibacterium propionicus (Pseudopropionibacterium propionicum), Proteus mirabilis, Proteus vulgaris, Providencia stuartii, Pseudomonas aeruginosa, Pseudomonas fluorescens, Pseudomonas putida, Pseudoramibacter alactolyticus, Ralstonia pickettii, Rothia aeria, Rothia dentocariosa, Rothia mucilaginosa, Scardovia inopinata, Schlegelella aquatica, Sebaldella termitidis, Selenomonas flueggei, Selenomonas noxia, Selenomonas sp. OT 478, Selenomonas sputigena, Serratia marcescens, Simonsiella muelleri, Slackia exigua, Solobacterium moorei, SR1 sp. OT 345, Staphylococcus aureus, Staphylococcus epidermidis, Staphylococcus saccharolyticus, Stenotrophomonas maltophilia, Streptococcus agalactiae, Streptococcus anginosus, Streptococcus australis, Streptococcus constellatus, Streptococcus cristatus, Streptococcus dentisani, Streptococcus gordonii, Streptococcus infantis, Streptococcus intermedius, Streptococcus milleri, Streptococcus mitis, Streptococcus mutans, Streptococcus oralis, Streptococcus parasanguinis, Streptococcus pneumoniae, Streptococcus pyogenes, Streptococcus salivarius, Streptococcus sanguinis, Streptococcus sobrinus, Streptococcus tigurinus, Streptococcus vestibularis, Tannerella forsythia, Treponema denticola, Treponema lecithinolyticum, Treponema medium, Treponema pallidum, Treponema socranskii, Treponema vincentii, Ureaplasma urealyticum, Veillonella atypica, Veillonella dispar, Veillonella parvula and Veillonella rogosae considered associated with human health are preferably considered as target nucleic acids, and rRNA genes of Actinomyces graevenitzii, Actinomyces israelii, Actinomyces naeslundii, Actinomyces odontolyticus, Actinomyces viscosus, Aggregatibacter actinomycetemcomitans, Aggregatibacter actinomycetemcomitans, Alloprevotella rava, Alloprevotella sp. OT 308, Campylobacter concisus, Campylobacter gracilis, Campylobacter rectus, Campylobacter showae, Capnocytophaga gingivalis, Capnocytophaga ochracea, Capnocytophaga sputigena, Corynebacterium matruchotii, Eikenella corrodens, Enterobacter cloacae, Enterococcus faecalis, Eubacterium nodatum, Eubacterium saphenum, Eubacterium sulci, Filifactor alocis, Fusobacterium nucleatum, Fusobacterium nucleatum subsp. animalis, Fusobacterium nucleatum subsp. polymorphum, Fusobacterium nucleatum subsp. vincentii, Fusobacterium periodonticum, Gemella sanguinis, Granulicatella adiacens, Haemophilus parainfluenzae, Helicobacter pylori, Klebsiella pneumoniae, Lactobacillus acidophilus, Lactobacillus casei, Lactobacillus salivarius, Leptotrichia sp. OT 215, Leptotrichia sp. OT 417, Leptotrichia sp. OT 462, Megasphaera micronuciformis, Neisseria flavescens, Parvimonas micra, Peptostreptococcus stomatis, Porphyromonas catoniae, Porphyromonas endodontalis, Porphyromonas gingivalis, Porphyromonas pasteri, Prevotella denticola, Prevotella histicola, Prevotella intermedia, Prevotella loescheii, Prevotella melaninogenica, Prevotella nigrescens, Prevotella pallens, Prevotella shahii, Prevotella sp. OT 306, Prevotella sp. OT 313, Prevotella veroralis, Rothia dentocariosa, Rothia mucilaginosa, Selenomonas noxia, Selenomonas sp. OT 478, Selenomonas sputigena, Solobacterium moorei, SR1 sp. OT 345, Staphylococcus aureus, Staphylococcus epidermidis, Streptococcus constellatus, Streptococcus dentisani, Streptococcus gordonii, Streptococcus intermedius, Streptococcus mitis, Streptococcus mutans, Streptococcus parasanguinis, Streptococcus pneumoniae, Streptococcus salivarius, Streptococcus sobrinus, Tannerella forsythia, Treponema denticola, Treponema medium, Treponema socranskii, Veillonella atypica, Veillonella parvula and Veillonella rogosae are more preferably considered as target nucleic acids but are not limited thereto.

### (2) Absolute Amount

The absolute amount of a target nucleic acid in the present invention means an amount of substance, the number of copies or a weight of the target nucleic acid. A result of NGS (read length) and a signal intensity of a DNA chip are relative quantitative values whereby respective abundance ratios or magnitudes in the values among samples can be compared but absolute amounts are not indicated.

### 2. Step (a)

### (1) Sample

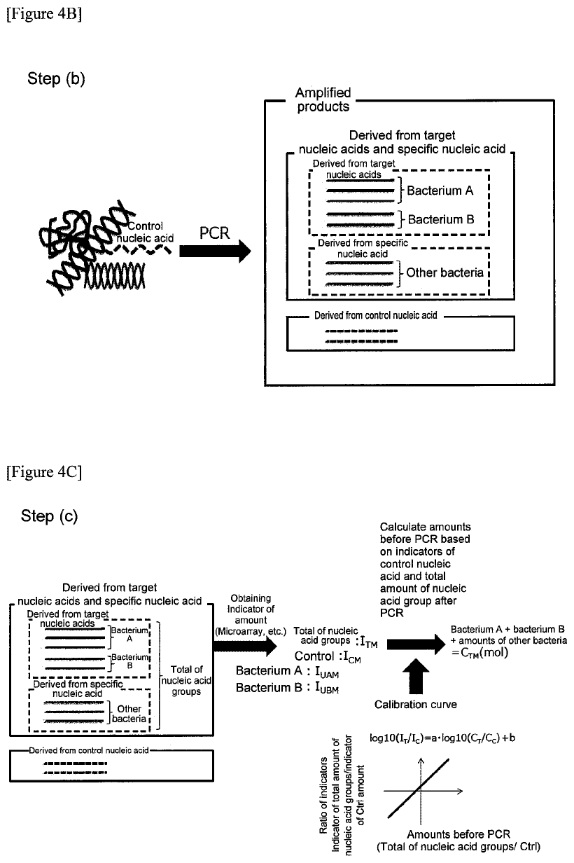

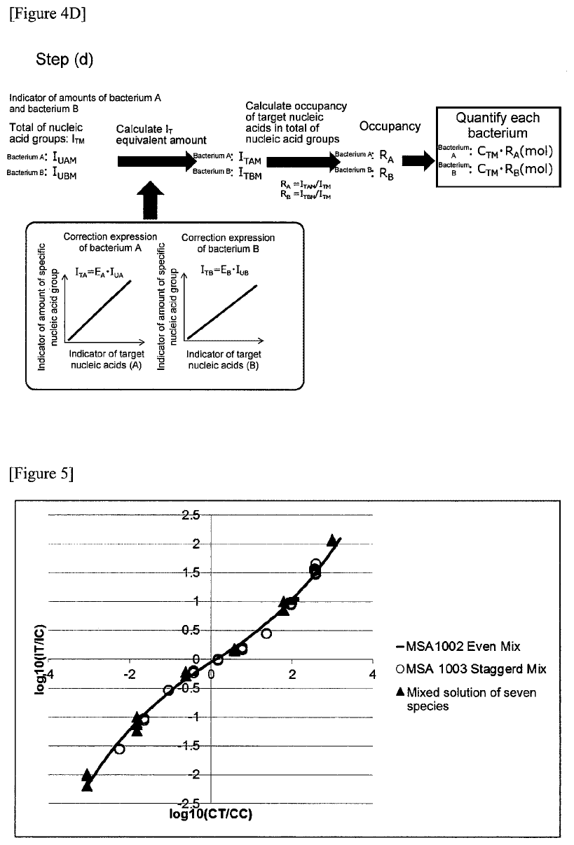

The method of the present invention comprises the step of (a) mixing a sample with a known amount of a control nucleic acid (FIG. 4A).

The sample in the present invention is not particularly restricted as long as nucleic acids are contained. For the sample, for example, foods and drinks, biological samples, environmental samples and samples from industrial process can be used.

Examples of the foods and drinks include naturally occurring foods and drinking waters, processed foods and processed drinks but are not limited thereto.

Examples of the biological sample are not limited and include tissues, cells, body fluids, plaques and feces obtained (for example, collected) from eucaryotes (for example, mammals, birds and plants). Examples of the mammal include mice, rats, rabbits, dogs, monkeys and human but are not limited thereto. The tissue is not limited as long as it can be obtained from various organs and examples include skin tissues, oral tissues, mucosal tissues, intestinal tissue and cancer tissues. The cell is not limited as long as it is a cell from these tissues and examples include cells from oral tissues, cells from mucosal tissues and cells from skin tissues. The body fluid is not limited and examples include saliva, gingival crevicular fluid (GCF), blood, urine, breast milk and amniotic fluid.

Examples of the environmental sample include soils, river waters, seawaters and hot spring waters.

Examples of the sample from the industrial process include active sludges.

Example of other samples include cultures of microorganisms artificially grown such as fermenter cultures.

In an embodiment, the sample of the present invention is at least one selected from the group consisting of active sludges, soils, river waters, seawaters, hot spring waters, drinking waters, processed foods, fermenter cultures, and tissues, cells and body fluids collected from eukaryotes.

In another embodiment, the sample of the present invention is at least one selected from the group consisting of saliva, plaque, gingival crevicular fluid (GCF), feces and skin-derived samples collected from mammals.

For the “sample” in the present invention, nucleic acids extracted from the samples described in the above examples and compositions containing extracted nucleic acids can be used in addition to the samples described in the above examples.

The method for extracting nucleic acids from a sample in the present invention is not limited and a known method can be used. For examples, a method of utilizing a commercial nucleic acid extraction kit and a method of phenol.chloroform extraction after treatment with proteinase K are included. When bacterial DNA is extracted, bacteriolysis treatment is included. Examples of the bacteriolysis treatment include enzyme treatment, heat treatment and bead crushing. A suspension of a sample can also be used directly for an amplification reaction.

A sample in the present invention can contain two or more types of target nucleic acids. The number of types of target nucleic acids contained in a sample has no particular upper limit and can be suitably selected in accordance with the purpose of evaluation.

### (2) Control Nucleic Acid

The control nucleic acid in the present invention is a nucleic acid added in a known amount to an amplification reaction solution before the reaction thereby to be a control when quantifying target nucleic acids and the like.

The addition of a control nucleic acid enables the determination of amounts before amplification from indicators of amounts after amplification of all target nucleic acids and the specific nucleic acid group to be collectively amplified with the target nucleic acids (hereinafter, “the specific nucleic acid group to be collectively amplified with the target nucleic acids” is also referred to as “the specific nucleic acid group”). For example, when the amplification by PCR method is used, an indicator of an amount dependent on an amount before amplification is obtained based on the principle of competitive PCR method.

The control nucleic acid is desirably a sequence which does not affect obtaining an indicator of an amount of a target nucleic acid and an indicator of the total amount of the target nucleic acid and a specific nucleic acid group and, for example, when a technique for obtaining an indicator of an amount is a DNA chip, a sequence which is not non-specifically hybridizable with a target nucleic acid or a probe for the target nucleic acid and the specific nucleic acid group is desirable, and more specifically, a specific sequence which has low identity with and similarity to a complementary sequence to a target nucleic acid and a probe for the target nucleic acid and a specific nucleic acid group is desirable.

The control nucleic acid needs to comprise a site required for amplification. For example, when the amplification by PCR method is used, the “site required for amplification” is a primer binding site. The base sequence of a primer binding site may be identical with or different from the base sequence of a primer binding site of a target nucleic acid.

It is difficult for the control nucleic acid to achieve the same amplification efficiency as those of a target nucleic acid and a specific nucleic acid group. However, on the other hand, it is desirable to bring the amplification efficiencies of the both closer as extremely different amplification efficiencies are presumed to affect the quantification. For achieving closer amplification efficiencies of a control nucleic acid, a target nucleic acid and the specific nucleic acid group, it is desirable that the base length of a control nucleic acid be designed so that there are no significant difference from base lengths at the sites to be amplified in a target nucleic acid and the specific nucleic acid group. For example, when amplified products from a target nucleic acid and the specific nucleic acid group are about 500 bp, amplified products of a control nucleic acid ranges desirably from about 300 bp to 1000 bp.

Examples of the design method for a base sequence of a control nucleic acid include a method in which a base sequence is randomly produced and a method of using a sequence having a partial homology with base sequences of a target nucleic acid and the specific nucleic acid group. The method of randomly producing a base sequence is particularly preferable in the present invention. This is because random designing is more likely to suppress the impact of the non-specific hybridization on quantification in the case of, for example, a DNA chip, in a sample in which the diversity of base sequences of target nucleic acids is high and the similarity thereof to each other is also high, such as bacterial florae in a human-derived sample.

Designing a control nucleic acid can be achieved by using, for example, RANDBETWEEN function in a Microsoft software “EXCEL” by which integers from 1 to 4 are randomly created in the X numbers (X is any number), linked to form X-digit numerical values composed only of the numerical values from 1 to 4 and are substituted with A for 1, T for 2, C for 3, and G for 4 thereby obtaining a large number of random sequences by X bases of ATGC. In these sequences, only the sequences having the same number of the sum of G and C as the sum of A and T are extracted, the extracted sequences are blast-searched on database such as NCBI GenBank, those having fewer analogous sequences are selected and, for example, in the case of amplifying by PCR method, sites required for amplification are added to both ends of the sequences thereby enabling the design. The sites required for amplification to be added at this time may be, for example, a sequence designed randomly as in the above or may be a sequence identical with the sequence at the primer binding site of a target nucleic acid. Additionally, the designed sequence can also be elongated by a suitable linkage or shortened by a partial removal.

The number of types of the control nucleic acid used in the present invention is not limited but the fewer the number thereof, the preferable from the viewpoint of simplicity, and is, for example, a single type.

### (3) Mixing

The “mixing” of a sample with a control nucleic acid in Step (a) includes the addition of the control nucleic acid to the sample or the addition of the sample to a composition containing the control nucleic acid.

### 3. Step (b)

(1) Specific Nucleic Acid Group to be Collectively Amplified with Target Nucleic Acids

The method of the present invention comprises the step of co-amplifying all target nucleic acids and the specific nucleic acid group to be collectively amplified with the target nucleic acids in a sample and a control nucleic acid (Step (b)) (FIG. 4B).

The “all target nucleic acids” in the present invention means all of the plural type of all target nucleic acids contained in a sample or amplified products.

In the present invention, a sample can contain a genome DNA of bacterial species to be measured, a genome DNA of bacterial species not to be measured and/or other nucleic acids (FIG. 4A). In the case where the genome DNA of bacterial species not to be measured contained in the sample comprise a common base sequence with a part of the base sequences of all target nucleic acids (for example, the 16SrRNA genes) (consensus sequence), when all target nucleic acids (for example, the 16SrRNA genes) in the sample or extracted nucleic acids are amplified, the 16SrRNA genes in the genome DNA of bacterial species not to be measured are also collectively amplified (FIG. 4B). Thus, a nucleic acid group other than target nucleic acids to be collectively (together with) amplified with all target nucleic acids are, in the present invention, referred to as “the specific nucleic acid group to be collectively amplified with target nucleic acids” or “the specific nucleic acid group”.

In a sample containing mRNA to be measured and mRNA not to be measured, for example, when the mRNA to be measured is considered as a target nucleic acid, in vitro transcription after reverse transcription utilizing a poly A sequence can be selected as the amplification method. In this case, the mRNA not to be measured are also collectively obtained but this mRNA also applies to “the specific nucleic acid group to be collectively amplified with target nucleic acids” or “the specific nucleic acid group”.

That is, “the specific nucleic acid group to be collectively amplified with target nucleic acids” (also referred to as “the specific nucleic acid group”) in the present invention refers to, other than target nucleic acids, a group of nucleic acids to be collectively amplified with the amplification of all target nucleic acids in a sample.

All target nucleic acids and the specific nucleic acid group in a sample are the nucleic acids to be collectively amplified all together as described above. Thus, the “all target nucleic acids and the specific nucleic acid group” in the present invention can be understood as a single collective group of nucleic acids comprising a common sequence. Additionally, in the present invention, a collective amount of all target nucleic acids and the specific nucleic acid group may refer to “a total of all target nucleic acids and the specific nucleic acid group.”

### (2) Co-Amplification

The “co-amplification” in the present invention means to amplify together in the same nucleic acid amplification reaction system.

The amplification technique of nucleic acids is not limited in the present invention and a known method can be used. When a target is DNA, examples include PCR method and LAMP (Loop-Mediated Isothermal Amplification) method, and when a target is RNA, examples include in vitro transcription after reverse transcription and PCR method. PCR method is particularly preferably utilized. When a DNA chip is used, for example, primers are fluorescence-labelled thereby to obtain an indicator of an amount as a signal intensity.

For example, when a bacterial 16SrRNA gene is amplified by PCR method, the amplification can be carried out under the conditions: an extracted DNA amount from a sample in the reaction solution of 1 pg to 10 ng, a control nucleic acid concentration of 0.0001 to 1 pM, respective primer concentrations of 0.01 to 10 μM, and a total solution volume of 1 to 100 μL. More preferably, the amplification can be carried out under the conditions: an extracted DNA amount from a sample in the reaction solution of 50 pg to 200 pg, a control nucleic acid concentration of 0.01 to 0.05 pM, respective primer concentrations of 0.1 to 2 μM, and a total solution volume of 20 μL. In this case, for example, the amplification can be carried out with a cycle number of 10 to 50. More preferably, a cycle number can be 15 to 40. The DNA polymerase used herein is not particularly restricted and a commercial heat-resistant DNA polymerase can be utilized. Similarly, the temperature, time, and the composition of a buffer solution are not particularly restricted and can be suitably set considering the DNA polymerase to be used and the size of amplification region of a nucleic acid of interest. When a commercial DNA polymerase is used in the quantification method of the present invention, the reaction time of the DNA polymerase is preferably set to be a sufficient reaction time of the recommended time or more described in an instruction manual. Specifically, the amplification reaction time of each step is preferably 1.2 times or more and 3 times or less the time recommended in an instruction manual as the reaction time of the DNA polymerase to be used. Further, in the final extension step, a so-called final extension time of 5 to 15 minutes can be set as needed. Thus, the complete length of a target nucleic acid and the like is homogeneously amplified.

When rRNA genes are quantified for the purpose of measuring the number of bacteria, the quantitative value can be divided (division) by the number of copies in 1 genome to obtain the number of bacteria as the rRNA genes are present in a plurality of copies in 1 genome. The number of copies of rRNA genes in 1 genome varies depending on bacterial species and known information (for example, Genbank) can be used for such a value.

When a DNA chip is used in the present invention, the step of co-amplification can comprise the step of enriching nucleic acids after amplification for single chains (single strands) hybridizable with probes loaded on a DNA chip. The present inventors found in the study for the first time that the hybridization of a single strand DNA rather than a double strand DNA enhances not only the sensitivity but also the accuracy thereof in the quantification method of the present invention.

The step of enriching for a single chain may be, for example, a step using the amplification reaction conditions for enriching for a single chain as the reaction conditions for co-amplification in the method of the present invention or a step further comprising treatment for enriching for a single chain after the reaction of co-amplification.

The amplification reaction conditions for enriching for a single chain include, for example, conditions to be so-called asymmetric PCR. Conditions to be asymmetric PCR can employ, specifically, conditions such as varied concentrations, different melting points, or different base lengths between a pair of primers used for PCR, singly or in combination. Further, in the PCR reaction composition, a concentration of a compound which affects a melting point of a nucleic acid (for example, a salt, dNTP, oligo, denaturating agent) is adjusted to make the condition more effective for enriching for a single chain. Additionally, in the asymmetric PCR, one primer of the pair can further be added during the PCR reaction or annealing conditions (temperatures or compound concentrations in the reaction solution) can be changed.

Examples of the treatment for enriching for a single chain after the reaction of co-amplification include a method for physically separating a double strand DNA after co-amplification to a single strand DNA (for example, denaturing high performance liquid chromatography) and a chemical method (for example, λ exonuclease treatment). Such a method is not particularly limited in the present invention but, from the viewpoint of operability, a method by λ exonuclease (Enzyme Commission numbers: EC 3.1.11.3) treatment is preferable.

In the case of carrying out this treatment step, one primer of a primer set used in the co-amplification of Step (b) phosphorylated at the 5′-end must be used. During the λ exonuclease reaction, the reaction conditions such as the reaction solution composition containing amplified fragments, buffer, and enzymes, reaction temperature, reaction time, and enzyme inactivation conditions are not particularly limited and, for example, the reaction conditions can be suitably set in accordance with the recommended condition of a commercial λ exonuclease. The λ exonuclease treatment can also be carried out directly without a purification step of the product after the co-amplification reaction in Step (b).

### 4. Step (c)

The present invention comprises the step of determining the total amount of all target nucleic acids and the specific nucleic acid group in a sample based on an indicator of the total amount of the amplified all target nucleic acids and the specific nucleic acid group and an indicator of an amount of the amplified control nucleic acid (Step (c)) (FIG. 4C). The “amplified all target nucleic acids and the specific nucleic acid group” and the “amplified control nucleic acid” herein respectively refer to, of the amplified products co-amplified as described above, the amplified products from all target nucleic acids and the specific nucleic acid group and the amplified products from the control nucleic acid.

This step preferably comprises the step of creating in advance a calibration curve of relationships, using a part or all of the target nucleic acids and the specific nucleic acid group and the control nucleic acid, between amounts before amplification and indicators of amounts after amplification of the total of a part or all of the target nucleic acids and the specific nucleic acid group and the control nucleic acid (Step (c1)). This is because the relationship between the total amount of all target nucleic acids and the specific nucleic acid group and the amount of the control nucleic acid changes before and after the amplification when the amplification efficiencies of the both vary.

In Patent Literature 1, for nucleic acids equivalent to a bacterial 16SrRNA gene and a control nucleic acid, an analysis is carried out in which a ratio of indicators of amounts after amplification is construed as a ratio of amounts before amplification or a value proportional thereto. However, such a construe is applicable only when amplification efficiencies of the both are the same. Additionally, in the control nucleic acid, an artificially designed sequence is used and it is difficult to bring its amplification efficiency to be the same as amplification efficiencies of all target nucleic acids and thus the presumption that a ratio of indicators of amounts after amplification is equivalent to a ratio of amounts before amplification is not most likely established.

In the present invention, a calibration curve is obtained in advance, and based on the calibration curve, the total amount of all target nucleic acids and the specific nucleic acid group is determined thereby enabling more accurate quantification.

The indicator of an amount herein refers to, for example, a signal intensity of a corresponding probe in the case of a DNA chip and, for example, a read length of a corresponding sequence in the case of NGS.

For nucleic acids used for creating a calibration curve in the present invention, a part or all of target nucleic acids and the specific nucleic acid group can be used. Hereinafter, the “a part or all of target nucleic acids and the specific nucleic acid group” used for creating a calibration curve is called “the nucleic acids for calibration.”

For the nucleic acids for calibration in the present invention, for example, when target nucleic acids are the 16SrRNA genes of two or more species of specific bacteria and the specific nucleic acid group is the bacterial 16SrRNA gene, a 16SrRNA gene comprised as a partial sequence in a genome DNA of a certain single bacterial species or a 16SrRNA gene comprised as a partial sequence in a mixture of genome DNA of a plurality of bacterial species can be used. The known sequence information on the full length of these bacterial 16SrRNA genes or a partial sequence comprising a site to be amplified is obtained and synthetic nucleic acids of these sequences can also be used as nucleic acids for calibration. The bacteria herein can be selected from bacteria having the 16SrRNA gene to be the target nucleic acid or can be selected from bacteria other than those. Alternatively, such a bacterium can be selected from both of them. That is, in the present invention, all of the nucleic acids to be target nucleic acids do not need to be used for creating a calibration curve and a part of the types of target nucleic acids and/or the specific nucleic acid group can be used. Thus, when the target nucleic acid contained in a sample are multiple types, a workload to create a calibration curve using all of these can be obviated thereby making the method simple.

Creation of a calibration curve is desirably carried out in a range wherein the concentration condition of the nucleic acids for calibration includes the total amount of all target nucleic acids and the specific nucleic group supposedly contained in a sample. For example, in the measurement of the 16SrRNA genes in saliva, when a sample is measured in such a way that 100 pg of the DNA extracted from the saliva is contained in an amplification reaction solution, an amount of substance of the 16SrRNA genes when the full 100 pg is supposedly bacterial genome DNA can be estimated. For example, given that the bacteria in saliva have an average genome molecular weight of 2.2×10⁶ and an average number of 16SrRNA copies in 1 genome of 4.5, an amount of substance of the 16SrRNA gene in 100 pg of the bacterial genome DNA is estimated about 0.31 amol. The average genome molecular weight of the bacteria in saliva and the average number of 16SrRNA copies in 1 genome used herein for the above estimation are values estimated by the present inventors from the average of data on the bacterial composition in saliva of healthy person reported in Oral Dis. 21, 748-54 (2015) and the information of each bacterium.

Additionally, according to Microbiome 6, 42 (2018) (https://doi.org/10.1186/s40168-018-0426-3), about 86% to 97% of the DNA extracted from saliva is human genome DNA, in other words, bacteria-derived DNA is estimated about 3% to 14%. Thus, the concentration condition of the nucleic acids for calibration in the reaction solution, when creating a calibration curve, can be determined so that the estimated concentration of bacteria-derived DNA is included. The calibration curve creation needs to be carried out under at least two or more concentration conditions. The maximum concentration condition in this case can be, for example, a concentration condition selected from 0.050 pM to 500 pM when an amplification reaction solution volume is 20 μL. It is more preferably a concentration condition selected from 0.050 pM to 50 pM. The minimum concentration condition can be, for example, a concentration condition selected from 0.10 aM to 0.10 fM when an amplification reaction solution volume is 20 μL. It is more preferably a concentration condition selected from 0.010 fM to 0.10 fM.

It is particularly preferable for the calibration curve to represent the relationships between [a ratio of (an indicator of the total amount of a part or all target nucleic acids and a specific nucleic acid group) to (an indicator of an amount of a control nucleic acid after amplification)] and [a ratio of (the total amount of a part or all target nucleic acids and the specific nucleic acid group) to (an amount of the control nucleic acid before amplification)]. These relationships are regression-analyzed and usable as a calibration curve. The simplest method is linear regression but other non-linear regression using non-linear models can also be used. When the amplification efficiencies of a control nucleic acid are the same as those of the total of a part or all target nucleic acids and the specific nucleic acid group are supposedly the same, this relational expression is represented by a linear expression. However, amplification efficiencies are often different in reality. Such a case plots a curved relationship but, for example, can be approximated by a linear expression when double logarithm is applied.

In other words, when an indicator of the total amount of amplified all target nucleic acids and the specific nucleic acid group is represented by I_{T}, an indicator of an amount of a control nucleic acid is represented by I_{C}, the total amount of all target nucleic acids and the specific nucleic acid group before amplification is represented by C_{T}, and an amount of the control nucleic acid before amplification is represented by C_{C}, the relationship of these values can be approximated by the expression below.

log₁₀(I_{T}÷I_{C})=a×log₁₀(C_{T}÷C_{C})+b (Expression 1)

The coefficients a and b can be determined from experiment data for creating a calibration curve by using, for example, LINEST function in a Microsoft software “EXCEL”.

Alternatively, a sigmoidal non-linear regression calibration curve can also be created. For example, when PCR method is used as the amplification reaction, both of the mechanism: an individual mechanism occurring partially between the total of all target nucleic acids and the specific nucleic acid group and a control nucleic acid and a mechanism occurring to the entire nucleic acids of the system, contribute to a saturation of amplification. Due to the combination of these mechanisms, [a ratio of (an indicator of the total amount of a part or all target nucleic acids and the specific nucleic acid group) to (an indicator of an amount of a control nucleic acid after amplification)] to changes of [a ratio of (the total amount of a part or all target nucleic acids and the specific nucleic acid group) to (an amount of the control nucleic acid before amplification)] changes in a non-monotonous manner. In this case, the use of a non-linear calibration curve enables highly accurate quantification in a wide concentration range.

The function is not particularly restricted as long as it gives a common sigmoidal curve and, for example, when an indicator of the total amount of amplified all target nucleic acids and the specific nucleic acid group is represented by I_{T}, an indicator of an amount of a control nucleic acid is represented by I_{C}, the total amount of all target nucleic acids and the specific nucleic acid group before amplification is represented by C_{T}, and an amount of the control nucleic acid before amplification is represented by C_{C}, the relationship of these values can be approximated by the expression below.

log 10(I_{T}÷I_{C})=b×(log 10(C_{T}÷C_{C})−c)÷(a−|log 10(C_{T}÷C_{C})−c|) (Expression 2)

The coefficients a, b, and c can be determined from experiment data for creating a calibration curve by regression using, for example, a tool called “SOLVER” in a Microsoft software “EXCEL”.

Descriptions of the symbols used in the present description are shown below.

I_{T}: Indicator of the total amount of amplified all target nucleic acids and the specific nucleic acid group (Intensity, Total bacteria)
I_{C}: Indicator of an amount of the amplified control nucleic acid (Intensity, Control)
C_{T}: Total amount of all target nucleic acids and the specific nucleic acid group before amplification (Concentration, Total bacteria)
C_{C}: An amount of a control nucleic acid before amplification (Concentration, Control)
C_{TM}: Measured value of the total amount of all target nucleic acids and the specific nucleic acid group before amplification (Concentration, Total bacteria, Measurement)
I_{TM}: Measured value of an indicator of the total amount of amplified all target nucleic acids and the specific nucleic acid group (Intensity, Total bacteria, Measurement)
I_{CM}: Measured value of an indicator of an amount of the amplified control nucleic acid

(Intensity, Control, Measurement)

I_{US}: Signal intensity of probe (x) from each target nucleic acid S (Intensity, Unique, Species) (S=A, B, . . . )
I_{TS}: Signal intensity of probe (y) from each target nucleic acid S (Intensity, Total bacteria, Species) (S=A, B, . . . )
E_{S}: Coefficient in each target nucleic acid S (Efficiency, Species) (S=A, B, . . . )
I_{TSM}: Measured value of a value equivalent to a signal intensity of probe (y) from each target nucleic acid S (Intensity, Total bacteria, Species, Measurement) (S=A, B, . . . )
I_{USM}: Measured value of a signal intensity of probe (x) corresponding to each target nucleic acid S (Intensity, Unique, Species, Measurement) (S=A, B, . . . )
R_{S}: Occupancy of each target nucleic acid S (Rate, Species) (S=A, B, . . . )

Step (c) in the present invention preferably comprises the step (Step (c2)) of calculating a total amount of all target nucleic acids and the specific nucleic acid group in a sample by applying an indicator of the total amount of the amplified all target nucleic acids and the specific nucleic acid group to be collectively amplified with the target nucleic acids from the sample and an indicator of an amount of the control nucleic acid to the calibration curve created in the previous step.

In this step, for example, when an indicator of the total amount of amplified all target nucleic acids and the specific nucleic acid group from the sample is represented by I_{TM} and an indicator of an amount of a control nucleic acid is represented by I_{CM} and are applied to the calibration curve created in the above Expression 1, a total amount of all target nucleic acids and the specific nucleic acid group before amplification C_{TM} (that is, in a sample) can be calculated using the expression below.

C_{TM}=C_{C}×10\hat{ }((log₁₀(I_{TM}÷I_{CM})−b)÷a) (Expression 3)

A sigmoidal calibration curve can also be calculated in the same manner when the indicators are applied to an approximate expression thereof. For example, when an indicator of the total amount of amplified all target nucleic acids and the specific nucleic acid group is represented by I_{TM} and an indicator of an amount of a control nucleic acid is represented by I_{CM} and are applied to the calibration curve created in the above Expression 2, a total amount of all target nucleic acids and the specific nucleic acid group before amplification C_{TM} (that is, in a sample) can be calculated using the expression below.

C_{TM}=C_{C}×10\hat{ }(a×log 10(I_{TM}÷I_{CM})÷(b+|log 10(I_{TM}÷I_{CM})|)+c) (Expression 4)

### 5. Step (d)

The present invention comprises the step of calculating an occupancy of each target nucleic acid in the total of all target nucleic acids and the specific nucleic acid group based on an indicator of the total amount of amplified all target nucleic acids and the specific nucleic acid group and an indicator of an amount of each amplified target nucleic acid thereby calculating an absolute amount of each target nucleic acid in a sample based on this occupancy (Step (d)) (FIG. 4D).

The “each target nucleic acid” in the present invention means a respective plurality types of target nucleic acids contained in a sample or amplified products.

In this step, an absolute amount of the total of all target nucleic acids and the specific nucleic acid group obtained in Step (c) is multiplied by an occupancy thereby to determine an absolute amount of each target nucleic acid in a sample.

The present invention comprises separately the step of determining the total amount of all target nucleic acids and the specific nucleic acid group from the step of determining an occupancy of each target nucleic acid. In the present invention, the occupancy of each target nucleic acid in the total of all target nucleic acids and the specific nucleic acid group is, for example, when the indicator of an amount is a read length of NGS, a ratio of a read length of the amplified product of each target nucleic acid to a read length of amplified products of all target nucleic acids and the specific nucleic acid group.

When a DNA chip is used in the present invention, the indicators of amounts are signal intensities of the following probes (x) to (z):

(x) a probe hybridizable respectively with amplified products of two or more types of target nucleic acids;

(y) a probe hybridizable in common with both amplified products of all target nucleic acids and a specific nucleic acid group in a sample; and

(z) a probe hybridizable with amplified products of the control nucleic acid.

As the probe (x) herein, a probe that is hybridizable specifically with respective amplified products of target nucleic acids is preferable.

Detailed description of a DNA chip will be described later.

In Step (d), the calculation of an occupancy is preferably carried out by the following Steps (d1) to (d3):

(d1) hybridizing two or more types of respective target nucleic acids (each target nucleic acid) with a DNA chip in advance and calculating a relational expression on signal intensities between the probe (x) and the probe (y) hybridizable with each target nucleic acid;

(d2) converting the signal intensity of the probe (x) obtained by hybridizing products amplified from a sample with the DNA chip to a value equivalent to the signal intensity of the probe (y) hybridizable with each target nucleic acid using the relational expression calculated in the above (d1); and

(d3) calculating a ratio of the value equivalent to the signal intensity of the probe (y) calculated in the above (d2) to the signal intensity of the probe (y) obtained by hybridizing products amplified from the sample with the DNA chip.

The target nucleic acid used herein in Step (d1) can be, for example, artificially synthesized nucleic acids or biological nucleic acids such as those extracted from an isolated bacterium as long as it is bacterial genome DNA.

A DNA chip, even with the same amount of nucleic acids to be hybridized, could cause different signal intensities depending on hybridization efficiencies when the type of probe varies or different various conditions. That is, the determination of an occupancy of each target nucleic acid in the total of all target nucleic acids and the specific nucleic acid group from the signal intensities of the probe (x) and the probe (y) requires the conversion of the signal intensity of the probe (x) to a value equivalent to the signal intensity of the probe (y) (Step (d2)). For such an achievement, a relational expression on the signal intensities between the probe (x) and the probe (y) needs to be obtained in advance in the above step of (d1). This relational expression can be suitably determined from experiments and can be, for example, approximated by a proportional relationship.

In other words, when a signal intensity of the probe (x) hybridizable with a single target nucleic acid S (S is an index of the type of each target nucleic acid) is represented by I_{US} and a signal intensity of the probe (y) is represented by I_{TS}, the relationship between I_{US} and I_{TS} can be approximated by, for example, the expression below.

I_{TS}=E_{S}×I_{US} (Expression 5)

This coefficient E_{S} can be determined from the signals of the respective probes obtained in Step (d1) using, for example, LINEST function in a Microsoft software “EXCEL”.

Here, when the signal intensity of the probe (x) corresponding to each target nucleic acid S obtained by hybridizing the products amplified from the sample with a DNA chip is represented by I_{USM} and the signal intensity of the probe (y) is represented by I_{TM}, the value equivalent to the signal intensity of the probe (y) from each target nucleic acid I_{TSM} to be determined in Step (d2) is represented by the expression below.

I_{TSM}=E_{S}×I_{USM} (Expression 6)

Further, here, the occupancy R_{S} of each target nucleic acid S can be calculated using the expression below (Step (d3)).

R_{S}=I_{TSM}÷I_{TM} (Expression 7)

In the present invention, Step (d) further comprises the step (Step (d4)) of calculating an absolute amount of each target nucleic acid in the sample by multiplying the total amount of all target nucleic acids and the specific nucleic acid group in the sample calculated in Step (c) by the occupancy calculated in Step (d3).

In this step, an absolute amount of each target nucleic acid in the sample can be calculated by multiplying the amount of the nucleic acid group calculated in Step (c) by the occupancy calculated in Step (d3).

### 6. DNA Chip

In the present invention, the DNA chip is not limited to any device as long as it is immobilized in such a way that the probes are independent and the position can be specified, and, as a wider dynamic range is demanded for bacterial florae analysis, an array having a large amount of probe immobilization is more suitable than a chip with probes immobilized simply on a planar substrate. Examples of such a DNA chip particularly include a fiber-type DNA chip on which probes are three-dimensionally immobilized through a gel.

The form of a support of a DNA chip is not particularly limited and any forms such as flat plates, rods, and beads can be used. When a flat plate is used as the support, predetermined probes by the type can be immobilized on a flat plate at a predetermined interval (spotting method and the like; see Science 270, 467-470 (1995) and the like). Additionally, predetermined probes can be successively synthesized by the type at a specific position on the flat plate (photolithography method and the like; see Science 251, 767-773 (1991) and the like).

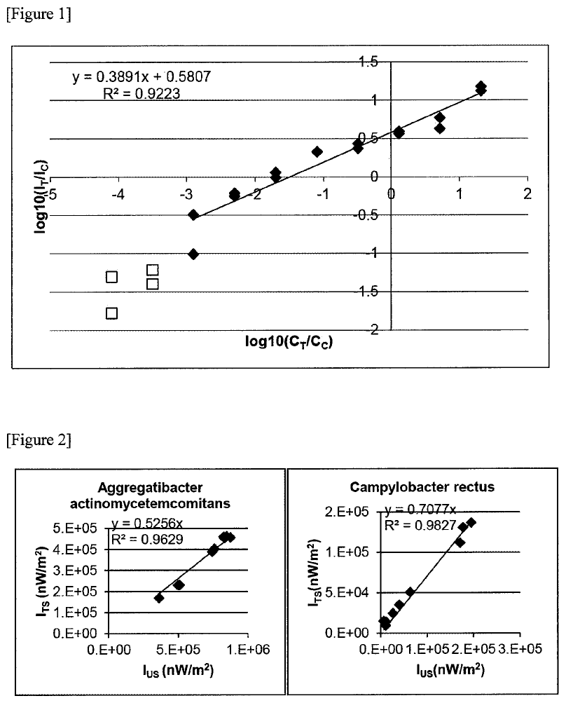

Examples of other preferable form of a support include those using hollow fibers. When a hollow fiber is used as the support, a preferable example includes a device obtained by immobilizing oligonucleotide probes by the type on each hollow fiber, bundling and immobilizing all hollow fibers and subsequently repeating cutting of the fibers in a longitudinal direction. This device can be described as the type of chip in which oligonucleotide probes are immobilized on a through-hole substrate (see FIG. 1 and the like of Japanese Patent No. 3510882).

The method for immobilizing probes on the substrate is not limited and can be any binding mode. Further, the method is not limited to direct immobilization on the support and, for example, a support is coated in advance with a polymer such as polylysine and probes can also be immobilized on the treated support. Furthermore, when a tubular body such as hollow fibers is used as the support, a gel-like substance is retained in the tubular body and probes can also be immobilized on the gel-like substance.

### 7. Probe

A probe captures a nucleic acid through hybridization and DNA hybridizable with a sequence specific to the nucleic acid to be captured can be used.

Typically, when a DNA chip collectively containing multiple types of probes is used, it is desirable to arrange the hybridization efficiencies among the probes as much as possible so that the multiple types of probes are subjected to the hybridization reaction under the same conditions. For this purpose, when designing probes, GC contents and sequence lengths must be adjusted and Tm values need to be arranged to be constant within a possible range. The type and number of probes loaded on a DNA chip are not particularly limited.

In the present invention, the sequence of a probe is preferably a sequence consisting of a complementary sequence to a specific base sequence to be captured.

In the present invention, the probe (the above probe (x)) hybridizable with respective amplified products of two or more types of target nucleic acids is preferably, for example, when capturing the 16SrRNA genes of Achromobacter xylosoxidans, Acinetobacter baumannii, Acinetobacter calcoaceticus, Acinetobacter lwoffii, Actinomyces graevenitzii, Actinomyces israelii, Actinomyces meyeri, Actinomyces naeslundii, Actinomyces odontolyticus, Actinomyces turicensis, Actinomyces viscosus, Aggregatibacter actinomycetemcomitans, Alloprevotella rava, Alloprevotella sp.OT 308, Atopobium parvulum, Bacteroides fragilis, Burkholderia cepacia, Campylobacter concisus, Campylobacter gracilis, Campylobacter rectus, Campylobacter showae, Capnocytophaga gingivalis, Capnocytophaga ochracea, Capnocytophaga sputigena, Chlamydophila (Chlamydia) pneumoniae, Chryseobacterium indologenes, Citrobacter freundii, Corynebacterium matruchotii, Corynebacterium striatum, Eikenella corrodens, Enterobacter aerogenes, Enterobacter cloacae, Enterobacter cloacae subsp. cloacae, Enterobacter cloacae subsp. dissolvens, Enterococcus faecium, Escherichia coli, Eubacterium nodatum, Eubacterium saphenum, Eubacterium sulci, Filifactor alocis, Fusobacterium necrophorum subsp. funduliforme, Fusobacterium necrophorum subsp. necrophorum, Fusobacterium nucleatum, Fusobacterium nucleatum subsp. animalis, Fusobacterium nucleatum subsp. polymorphum, Fusobacterium nucleatum subsp. vincentii, Fusobacterium periodonticum, Gemella sanguinis, Granulicatella adiacens, Haemophilus influenzae, Haemophilus parainfluenzae, Helicobacter pylori, Klebsiella pneumoniae subsp. pneumoniae, Lactobacillus acidophilus, Lactobacillus casei, Lactobacillus salivarius, Legionella pneumophila, Legionella pneumophila subsp. pneumophila str. Philadelphia, Leptotrichia sp. OT 215, Leptotrichia sp. OT 417, Leptotrichia sp. OT 462, Megasphaera micronuciformis, Moraxella catarrhalis, Morganella morganii, Mycobacterium avium, Mycobacterium intracellulare, Mycobacterium kansasii, Mycoplasma pneumoniae, Neisseria flavescens, Nocardia brasilliensis, Parvimonas micra, Peptostreptococcus stomatis, Porphyromonas catoniae, Porphyromonas endodontalis, Porphyromonas gingivalis, Porphyromonas pasteri, Prevotella dentalis, Prevotella denticola, Prevotella histicola, Prevotella intermedia, Prevotella loescheii, Prevotella melaninogenica, Prevotella nigrescens, Prevotella oris, Prevotella pallens, Prevotella shahii, Prevotella veroralis, Propionibacterium acnes (Cutibacterium acnes), Proteus mirabilis, Proteus vulgaris, Pseudomonas aeruginosa, Rothia dentocariosa, Rothia mucilaginosa, Selenomonas noxia, Selenomonas sp. OT 478, Selenomonas sputigena, Serratia marcescens, Serratia marcescens subsp. marcescens, Solobacterium moorei, SR1 sp. OT 345, Staphylococcus aureus, Staphylococcus epidermidis, Stenotrophomonas maltophilia, Streptococcus agalactiae, Streptococcus anginosus, Streptococcus constellatus, Streptococcus dentisani, Streptococcus gordonii, Streptococcus intermedius, Streptococcus mitis, Streptococcus mutans, Streptococcus parasanguinis, Streptococcus pneumoniae, Streptococcus pyogenes, Streptococcus salivarius, Streptococcus sobrinus, Tannerella forsythia, Treponema denticola, Treponema medium, Treponema socranskii, Veillonella atypica, Veillonella parvula, Veillonella rogosae, and the genera of Streptococcus, a sequence selected from the group consisting of the base sequences as set forth in SEQ ID NO: 3 to 191.

In this case, such a probe can be a complementary sequence to the sequence selected from the group consisting of the base sequences as set forth in SEQ ID NO: 3 to 191 or can be a sequence substantially identical with the sequence selected from the group consisting of the base sequences as set forth in SEQ ID NO: 3 to 191 or a complementary sequence thereto.

The substantially identical sequence herein refers to a sequence hybridizable under stringent condition with the sequence selected from the group consisting of the base sequences as set forth in SEQ ID NO: 3 to 191 or a complementary sequence thereto.

The length of probe is, for example, a sequence of 13 bases or more, preferably 15 bases or more, and more preferably 17 bases or more. The stringent condition herein means a condition under which a specific hybrid is formed and a non-specific hybrid is not formed. That is, the stringent condition refers to a condition under which a pair of polynucleotides having a high homology (a homology or identity of 95% or more, preferably 96% or more, more preferably 97% or more, further preferably 98% or more, and most preferably 99% or more) is hybridized.

The stringent condition in the present invention can be any of a low stringent condition, a moderate stringent condition, and a high stringent condition. The “low stringent condition” is a condition of, for example, 5×SSC, 5×Denhardt's solution, 0.5% SDS, 50% formamide, and 32° C. The “moderate stringent condition” is a condition of, for example, 5×SSC, 5×Denhardt's solution, 0.5% SDS, 50% formamide, and 42° C. The “high stringent condition” is a condition of, for example, 5×SSC, 5×Denhardt's solution, 0.5% SDS, 50% formamide, and 50° C. Under these conditions, at a higher temperature, it can be expected to efficiently capture a DNA having a higher homology. It is conceivable that a plurality of elements affect the stringency of hybridization such as temperature, probe concentration, probe length, ion intensity, time, and salt concentration but a person skilled in the art can achieve the equivalent stringency by suitably selecting these elements. Detailed procedures of hybridization method can be referred to “Molecular Cloning, A Laboratory Manual (4th edition)” (Cold Spring Harbor Laboratory Press (2012)) and the like.

Further, for the stringent condition, conditions employed by well-known routine techniques in the filed such as Northern blotting method, dot blot method, colony hybridization method, plaque hybridization method, or Southern blotting hybridization method can be set.

In the present invention, the probe (the above probe (y)) hybridizable in common with both amplified products of all target nucleic acids and the specific nucleic acid group is preferably a complementary sequence to a sequence comprised commonly in the amplified products of the sequences comprised in all target nucleic acids and the specific nucleic acid group. For example, the probe, when capturing bacterial 16SrRNA genes, can be a part of a complementary strand of a consensus sequence of the bacterial 16SrRNA genes. Examples of the probe in this case include a probe comprising the base sequence as set forth in SEQ ID NO: 2.

In the present invention, the probe (the above probe (z)) hybridizable with amplified products of the control nucleic acid is preferably a part of a complementary sequence of amplified products of the control nucleic acid. For example, when the base sequence as set forth in SEQ ID NO: 196 is used as the sequence of a control nucleic acid, examples of the probe include a probe comprising the base sequence as set forth in SEQ ID NO: 1.

### 8. Kit

The present invention provides a kit comprising, for achieving the above quantitative technique, one or two pairs of primer sets for co-amplifying all target nucleic acids and the specific nucleic acid group and a control nucleic acid by PCR method, a control nucleic acid, and a DNA chip on which the above probes (x) to (z) are loaded.

When the target nucleic acids are the 16SrRNA genes of, for example, Achromobacter xylosoxidans, Acinetobacter baumannii, Acinetobacter calcoaceticus, Acinetobacter lwoffii, Actinomyces graevenitzii, Actinomyces israelii, Actinomyces meyeri, Actinomyces naeslundii, Actinomyces odontolyticus, Actinomyces turicensis, Actinomyces viscosus, Aggregatibacter actinomycetemcomitans, Alloprevotella rava, Alloprevotella sp.OT 308, Atopobium parvulum, Bacteroides fragilis, Burkholderia cepacia, Campylobacter concisus, Campylobacter gracilis, Campylobacter rectus, Campylobacter showae, Capnocytophaga gingivalis, Capnocytophaga ochracea, Capnocytophaga sputigena, Chlamydophila (Chlamydia) pneumoniae, Chryseobacterium indologenes, Citrobacter freundii, Corynebacterium matruchotii, Corynebacterium striatum, Eikenella corrodens, Enterobacter aerogenes, Enterobacter cloacae, Enterobacter cloacae subsp. cloacae, Enterobacter cloacae subsp. dissolvens, Enterococcus faecium, Escherichia coli, Eubacterium nodatum, Eubacterium saphenum, Eubacterium sulci, Filifactor alocis, Fusobacterium necrophorum subsp. funduliforme, Fusobacterium necrophorum subsp. necrophorum, Fusobacterium nucleatum, Fusobacterium nucleatum subsp. animalis, Fusobacterium nucleatum subsp. polymorphum, Fusobacterium nucleatum subsp. vincentii, Fusobacterium periodonticum, Gemella sanguinis, Granulicatella adiacens, Haemophilus influenzae, Haemophilus parainfluenzae, Helicobacter pylori, Klebsiella pneumoniae subsp. pneumoniae, Lactobacillus acidophilus, Lactobacillus casei, Lactobacillus salivarius, Legionella pneumophila, Legionella pneumophila subsp. pneumophila str. Philadelphia, Leptotrichia sp. OT 215, Leptotrichia sp. OT 417, Leptotrichia sp. OT 462, Megasphaera micronuciformis, Moraxella catarrhalis, Morganella morganii, Mycobacterium avium, Mycobacterium intracellulare, Mycobacterium kansasii, Mycoplasma pneumoniae, Neisseria flavescens, Nocardia brasilliensis, Parvimonas micra, Peptostreptococcus stomatis, Porphyromonas catoniae, Porphyromonas endodontalis, Porphyromonas gingivalis, Porphyromonas pasteri, Prevotella dentalis, Prevotella denticola, Prevotella histicola, Prevotella intermedia, Prevotella loescheii, Prevotella melaninogenica, Prevotella nigrescens, Prevotella oris, Prevotella pallens, Prevotella shahii, Prevotella veroralis, Propionibacterium acnes (Cutibacterium acnes), Proteus mirabilis, Proteus vulgaris, Pseudomonas aeruginosa, Rothia dentocariosa, Rothia mucilaginosa, Selenomonas noxia, Selenomonas sp. OT 478, Selenomonas sputigena, Serratia marcescens, Serratia marcescens subsp. marcescens, Solobacterium moorei, SR1 sp. OT 345, Staphylococcus aureus, Staphylococcus epidermidis, Stenotrophomonas maltophilia, Streptococcus agalactiae, Streptococcus anginosus, Streptococcus constellatus, Streptococcus dentisani, Streptococcus gordonii, Streptococcus intermedius, Streptococcus mitis, Streptococcus mutans, Streptococcus parasanguinis, Streptococcus pneumoniae, Streptococcus pyogenes, Streptococcus salivarius, Streptococcus sobrinus, Tannerella forsythia, Treponema denticola, Treponema medium, Treponema socranskii, Veillonella atypica, Veillonella parvula, Veillonella rogosae, and the genera of Streptococcus, the probe (x) is a sequence selected from the group consisting of the base sequences as set forth in SEQ ID NO: 3 to 191. In this case, the probe can be a complementary sequence to the sequence selected from the group consisting of the base sequences as set forth in SEQ ID NO: 3 to 191 or can be a sequence substantially identical with the sequence selected from the group consisting of the base sequences as set forth in SEQ ID NO: 3 to 191 or a complementary sequence thereto. The “sequence substantially identical with” is as defined as described above.

Additionally, the present invention provides a DNA chip on which the above probes (x) to (z) are loaded, wherein the probe (x) is a sequence selected from the group consisting of the base sequences as set forth in SEQ ID NO: 43 to 191, a complementary sequence to the sequence selected from the group consisting of the base sequences as set forth in SEQ ID NO: 43 to 191, or a sequence substantially identical with the sequence selected from the group consisting of the base sequences as set forth in SEQ ID NO: 43 to 191 or a complementary sequence thereof.

The probes used in the present invention are shown in Tables 1 below.

TABLE 1

| SEQ ID | | |
|---|---|---|
| NO | Name | Probe sequence |
| 1 | Probe for control nucleic acid | CTATTCGACCAGCGATATCACTACGTAGGC |
| 2 | Probe for total 16SrRNA genes | CGTATTACCGCGGCTGCTGGCAC |
| 3 | Probe for Aggregatibacter | GTCAATTTGGCATGCTATTAACACACC |
| | actinomycetemcomitans | |
| 4 | Probe for Aggregatibacter | GTCAAGTTGGCATGCTATTAACACACC |
| | actinomycetemcomitans | |
| 5 | Probe for Campylobacter rectus | GTCATAATTCTTTCCCAAGA |
| 6 | Probe for Capnocytophaga gingivalis | TACACGTACACCTTATTCTT |
| 7 | Probe for Fusobacterium nucleatum | TACATTCCGAAAAACGTCAT |
| 8 | Probe for Porphyromonas gingivalis | TTCAATGCAATACTCGTATC |
| 9 | Probe for Prevotella intermedia | CGAAGGGTAAATGCAAAGGGGC |
| 10 | Probe for Prevotella intermedia | CGAAGGGTAAATGCAAAAAGGC |
| 11 | Probe for Prevotella nigrescens | CTTTATTCCCACATAAAAGC |
| 12 | Probe for Selenomonas noxia | TTCGCATTAGGCACGTTC |
| 13 | Probe for Streptococcus constellatus | AAGTACCGTCACTGTGTG |
| 14 | Probe for Streptococcus gordonii | CACCCGTTCTTCTCTTACA |
| 15 | Probe for Streptococcus intermedius | ACAGTATGAACTTTCCATTCT |
| 15 | Probe for Tannerella forsythia | CACGTATCTCATTTTATTCCCCTGT |
| 17 | Probe for Treponema denticola | CCTCTTCTTCTTATTCTTCATCTGC |
| 18 | Probe for Veillonella parvula | TCCTTCTAACTGTTCGC |
| 19 | Probe for Actinomyces odontolyticus | AAGTCAGCCCGTACCCA |
| 20 | Probe for Actinomyces viscosus | CCACCCACAAGGAGCAG |
| 21 | Probe for Aggregatibacter | GTCAATTTGGCATGCTATTA |
| | actinomycetemcomitans | |
| 22 | Probe for Campylobacter concisus | CCCAAGCAGTTCTATGGT |
| 23 | Probe for Campylobacter gracilis | GCCTTCGCAATAGGTATT |
| 24 | Probe for Campylobacter showae | CAATGGGTATTCTTCTTGAT |
| 25 | Probe for Capnocytophaga ochracea | CAACCATTCAAGACCAACA |
| 25 | Probe for Capnocytophaga sputigena | TCAAAGGCAGTTGCTTAGT |
| 27 | Probe for Eikenella corrodens | CTCTAGCTATCCAGTTCAG |
| 28 | Probe for Fusobacterium nucleatum | TTTCTTTCTTCCCAACTGAA |
| | subsp. animalis | |
| 29 | Probe for Fusobacterium nucleatum | CCAGTACTCTAGTTACACA |
| | subsp. polymorphum | |
| 30 | Probe for Fusobacterium nucleatum | TAGTTATACAGTTTCCAACG |
| | subsp. vincentii | |
| 31 | Probe for Fusobacterium periodonticum | TATGCAGTTTCCAACGCAA |
| 32 | Probe for Lactobacillus salivarius | GCCGTGACTTGCTGGTT |
| 33 | Probe for Lactobacillus casei | CTGTCCTCTTCTGCACT |
| 34 | Probe for Prevotella intermedia | GGGTAAATGCAAAAAGGCA |
| 35 | Probe for all Streptococcus | TTAGCCGTCCCTTTCTGG |
| 36 | Probe for Streptococcus dentisani | TCCCCTCTTGCACTCAAGT |
| 37 | Probe for Streptococcus mitis | TCTCCCCTCTTGCACTCA |
| 38 | Probe for Streptococcus mutans | CACACGTTCTTGACTTAC |
| 39 | Probe for Streptococcus sobrinus | CCGTCACTGTGTAAGCTT |
| 40 | Probe for Tannerella forsythia | CACGTATCTCATTTTATTCC |
| 41 | Probe for Treponema denticola | CCTCTTCTTCTTATTCTTCAT |
| 42 | Probe for Veillonella parvula | TATTCGCAAGAAGGCCTT |
| 43 | Probe for Actinomyces graevenitzii | AAAAAGCAGTGCCTTGTTCC |
| 44 | Probe for Actinomyces israelii | GCGCTTCATAACCCGGCTAC |
| 45 | Probe for Actinomyces naeslundii | AACCCACCCACAAACGA |
| 46 | Probe for Alloprevotella rava. | TTCCCAACTAAAAGCAGTTTA |
| | Alloprevotella sp. OT 308 | |
| 47 | Probe for Corynebacterium matruchotii | TCTTAACAAAGGTACCGTCACC |
| 48 | Probe for Eubacterium nodatum | CCTACGCTTACTTAACCACCTA |
| 49 | Probe for Eubacterium saphenum | CCCTAGGACAGAGGCTTACA |
| 50 | Probe for Eubacterium sulci | AAACCCTGCGCTTAAGGTGC |
| 51 | Probe for Filifactor alocis | CCCCTACTACAGAGTTTTACGA |
| 52 | Probe for Gamella sanguinis | CCGTCTCTACTGTATATAGT |
| 53 | Probe for Granulicatella adiacens | GTCAAGGCGCTAACAGTTAC |
| 54 | Probe for Haemophilus parainfluenzae | AGTTAACGTCAATCACCTAG |
| 55 | Probe for Helicobacter pylori | GCGGGATAGTCAGTCAGGTG |
| 56 | Probe for Lactobacillus acidophilus | CAGTTTCCGATGCAGTTCC |
| 57 | Probe for Leptotrichia spp. | CGTGCAGTTCCGTCCACCTC |
| | (OT 215,417,462) | |
| 58 | Probe for Megasphaera micronuciformis | TAACCACAAGATTATTCGTC |
| 59 | Probe for Neisseria flavescens | AGCTGTCGATATTAGCAACAG |
| 60 | Probe for Parvimonas micra | GTGCTTAATGAGGTTAAGCC |
| 61 | Probe for Peptostreptococcus stomatis | ACCACCGACTTGAAGGACCA |
| 62 | Probe for Porphyromonas catoniae | GGTACATTCACTATGGTACACG |
| 63 | Probe for Porphyromonas endodontalis | TACATGCATCTCAGCTACACGT |
| 64 | Probe for Porphyromonas pasteri | ACACGTGACTCTTGTTATTC |
| 65 | Probe for Prevotella denticola | AGTCAGACGTTGGGCGCCTA |
| 66 | Probe for Prevotella histicola | CACGTGACTGACTTTATCCC |
| 67 | Probe for Prevotella loescheii | CCTACTTTCAGCGCACTCAA |
| 68 | Probe for Prevotella melaninogenica | AATAGGGACACGTCCCTAAC |
| 69 | Probe for Prevotella pallens | CACGTGCATCAAATTATTCTCG |
| 70 | Probe for Prevotella shahii | ACGTGGGCTCTTTTATCCCC |
| 71 | Probe for Prevotella veroralis | ACACGTGATTGACTTTATCC |
| 72 | Probe for Rothia dentocariosa | ACCCACTGCAAAACCAGGGT |
| 73 | Probe for Rothia mucilaginosa | TCTCTTCTTCCCTGCTAACA |
| 74 | Probe for Selenomonas sp. OT 478 | ACTATTCGCACTAGGCACGT |
| 75 | Probe for Selenomonas sputigena | GTACCGTCACCCAAACTCAATA |
| 76 | Probe for Solobacterium moorei | CCAACAATTTAACCACTTAC |
| 77 | Probe for SR1 sp. OT 345 | CGTCATTCGTCTTCTGCCAA |
| 78 | Probe for Streptococcus parasanguinis | CTGGTAAGTTACCGTCAC |
| 79 | Probe for Streptococcus salivarius | CACACTCGTTCTTGACTTAC |
| 80 | Probe for Streptococcus sobrinus | TACACACGTTCTTCCCCTAC |
| 81 | Probe for Treponema medium | GTCGATTACCGTCATCAGATG |
| 82 | Probe for Treponema socranskii | TTCCTCCAAAACTTATTCCT |
| 83 | Probe for Veillonella atypica | CGTCAAATCCTCGCACTATTC |
| 84 | Probe for Veillonella rogosae | ACCGTCAATTCCTCTAACTATT |
| 85 | Probe for Achromobacter xylosoxidans | CAGTTTCGCGAGGTATTAAC |
| 86 | Probe for Achromobacter xylosoxidans | CACCAGACGAAGTCCGTGCT |
| 87 | Probe for Acinetobacter baumannii | CTAGGTCCGGTAGCAAGCTACC |
| 88 | Probe for Acinetobacter baumannii | TACCTTCCCCCGCTCGACTT |
| 89 | Probe for Acinetobacter calcoaceticus | ATCAGTAGCAAGCTACCTCTCTC |
| 90 | Probe for Acinetobacter calcoaceticus | CGCTAAGATCAGTAGCAAGCTA |
| 91 | Probe for Acinetobacter lwoffii | GTATTAATCTCGGTAGCCTCC |
| 92 | Probe for Acinetobacter lwoffii | CTAGGTTAGGTAGCAAGCTACATT |
| 93 | Probe for Actinomyces israelii | CACCAAAACACCACAAAAGTGA |
| 94 | Probe for Actinomyces israelii | ACTAACCCACCCCGCAAAAA |
| 95 | Probe for Actinomyces meyeri | TCCAGTATTAGCACCCATTT |
| 96 | Probe for Actinomyces odontolyticus | ATGCGAAGATCAGTGAATATCC |
| 97 | Probe for Actinomyces turicensis | AAGTTGGAGCATCATCGTTC |
| 98 | Probe for Actinomyces turicensis | GCCAATGAGTGAATATCCAG |
| 99 | Probe for Atopobium parvulum | GGAAGTCTCGAAGTATTCGGT |
| 100 | Probe for Atopobium parvulum | GAGCTTTCTCGAGTCTTCCATG |
| 101 | Probe for Bacteroides fragilis | CGGAATCATTATGCTATCGGGT |
| 102 | Probe for Bacteroides fragilis | CAAGCTTTCTTCCTGATGCC |
| 103 | Probe for Burkholderia cepacia | TAGAGCCAAGGATTTCTTTCCG |
| 104 | Probe for Burkholderia cepacia | ACTACAGGACATGTTCCGAT |
| 105 | Probe for Chryseobacterium | CTCTCAAGATCCCGAAAGATC |
| | indologenes | |
| 106 | Probe for Chryseobacterium | GTACGCCGCTCTCAAGATCC |
| | indologenes | |
| 107 | Probe for Citrobacter freundii | TAACCACAACGCCTTCCTCC |
| 108 | Probe for Citrobacter freundii | GTCACCCAAGGAGCAAGCTC |
| 109 | Probe for Corynebacterium striatum | CTGACACTAAACAGTGGTCCTA |
| 110 | Probe for Corynebacterium striatum | TCGAGTACCCGCAGCAAGCT |
| 111 | Probe for Eikenella corrodens | CGGACAGTAGTGCAAGCACT |
| 112 | Probe for Eikenella corrodens | AATAACGCGAGGTCTTGCGA |
| 113 | Probe for Enterobacter aerogenes | TTAACCTTAACGCCTTCCTC |
| 114 | Probe for Enterobacter aerogenes | CCTCGCTGAAAGTACTTTACAA |
| 115 | Probe for Enterobacter cloacae subsp. | CGGTTATTAACCACAACACC |
| | cloacae | |
| 116 | Probe for Enterobacter cloacae | AGCTCTCTGTGCTACCGTTC |
| 117 | Probe for Enterobacter cloacae | TCTCTGTGCTACCGTTCGAC |
| 118 | Probe for Enterobacter cloacae subsp. | TCTTTGGTCTTGCGACGTTA |
| | dissolvens | |
| 119 | Probe for Enterococcus faecium | CTCTCAGGTGCGGCTATGCA |
| 120 | Probe for Escherichia coli | AGGTCCCCCTCTTTGTGCTT |
| 121 | Probe for Escherichia coli | CGTCAATGAGCAAAGGTATT |
| 122 | Probe for Fusobacterium necrophorum | GACATACGTAATTACCGCGG |
| | subsp. rum | |
| 123 | Probe for Fusobacterium necrophorum | GCTTGCGACATACGTAATTA |
| | subsp. rum | |
| 124 | Probe for Fusobacterium necrophorum | ATGTTGTCCCGGTCTAAGAG |
| | subsp. rne | |
| 125 | Probe for Legionella pneumophila | CTTCAAGGCATATTCCTACG |
| 126 | Probe for Legionella pneumophila | TTACAACCCTCAGGCCTTCT |
| 127 | Probe for Legionella pneumophila | TTACAACCCTCAGGCCTTCTT |
| 128 | Probe for Legionella pneumophila | CCICTICAAGGCATATTCCTAC |
| 129 | Probe for Legionella pneumophila | CAGTATTATCTGACCGTCCC |
| 130 | Probe for Legionella pneumophila | AGGCTAATCTTAAAGCGCCAGG |
| 131 | Probe for Legionella pneumophila subsp. | CAGCTTTCGTCCTCAGACATT |
| | pneumophila str. Philadelphia | |
| 132 | Probe for Moraxella catarrhalis | GCTTCCTAACTTCGTTCGAC |
| 133 | Probe for Moraxella catarrhalis | GTCAGGGCTTATGGGTATTA |
| 134 | Probe for Morganella morganii | ACATCTGACTCAATCAACCGC |
| 135 | Probe for Morganella morganii | TATTAACCTTGACACCTTCCTC |
| 136 | Probe for Mycobacterium avium | CAGAAGACATGCGTCTTGAG |
| 137 | Probe for Mycobacterium avium | GAGTACCTCCGAAGAGGCCTTT |
| 138 | Probe for Mycobacterium intracellulare | ATTGCCCACGTGTTACTCAC |
| 139 | Probe for Mycobacterium intracellulare | CTTCTTCTCCACCTACCGTC |
| 140 | Probe for Mycobacterium kansasii | CCACTCGAGTGTCTCCGAAG |
| 141 | Probe for Mycobacterium kansasii | AGGCTTATCCCGGTGTGCAG |
| 142 | Probe for Nocardia brasilliensis | ACCCAACAGCATGCACTGAAA |
| 143 | Probe for Nocardia brasNiensis | GTCAGTTACTTCCCAGAGAC |
| 144 | Probe for Peptostreptococcus stomatis | CCGCTTGGTTTTGCTCAGAATT |
| 145 | Probe for Peptostreptococcus stomatis | ACTCAATACTGAGCAAACCCTC |
| 145 | Probe for Prevotella dentalis | GAGGACCTCGTTGGAATACATA |
| 147 | Probe for Prevotella dentalis | CTGCTTGAAGGTAGGTTGGATA |
| 148 | Probe for Prevotella melaninogenica | CCCTAACTTTATCCCCATACA |
| 149 | Probe for Prevotella melaninogenica | GTATGGTACCTGCAAATAGG |
| 150 | Probe for Prevotella oris | CTTTAATCGTCGTTGGATGCC |
| 151 | Probe for Prevotella oris | AGACGATGCCATGAGGTATT |
| 152 | Probe for Propionibacterium acnes | TCTTTACCCATTACCGTCAC |
| | (Cutibacterium acnes) | |
| 153 | Probe for Propionibacterium acnes | CCCAAGATTACACTTCCGAC |
| | (Cutibacterium acnes) | |
| 154 | Probe for Proteus mirabilis | GATTAACGCCTGCACCCTCC |
| 155 | Probe for Proteus mirabilis | CGCCTGCACCCTCCGTATTA |
| 156 | Probe for Proteus vulgaris | TACCGCTCGATTGCATGTGT |
| 157 | Probe for Proteus vulgaris | AACTTTATCACCTTCCTCCC |
| 158 | Probe for Staphylococcus epidermidis | GCGGTTCAATATATTATCCG |
| 159 | Probe for Staphylococcus epidermidis | ATAAGTGACAGCAAAACCGT |
| 160 | Probe for Stenotrophomonas maltophilia | TTAGCCAGCTGGATTTCTTT |
| 161 | Probe for Stenotrophomonas maltophilia | CGACAGAGTAGATTCCGATG |
| 162 | Probe for Streptococcus agalactiae | AGTCTAGTGTAAACACCAAACCTC |
| 163 | Probe for Streptococcus agalactiae | ACTCCTACCAACGTTCTTCTC |
| 164 | Probe for Streptococcus anginosus | CATCTACTAGCGATGCAATTG |
| 165 | Probe for Streptococcus anginosus | TTAAGTACCGTCACAGCATG |
| 166 | Probe for Veillonella atypica | ACCTTTCATCCAGTCTCGAT |
| 167 | Probe for Veillonella atypica | CGTCAAATCCTCGCACTATT |
| 168 | Probe for Helicobacter pylori | CACACCTGACTGACTATCCC |
| 169 | Probe for Helicobacter pylori | CCACTAATCAGCACTCTAGCA |
| 170 | Probe for Helicobacter pylori | AAACTAAGAGGCACATGACC |
| 171 | Probe for Chlamydophila (Chlamydia) | GTTTTAAATGCTGACTTGGGG |
| | pneumoniae | |
| 172 | Probe for Chlamydophila (Chlamydia) | GCATAAACTCTTCCTCAACC |
| | pneumoniae | |
| 173 | Probe for Chlamydophila (Chlamydia) | AACCGTTATCCCCAAGTTGA |
| | pneumoniae | |
| 174 | Probe for Haemophilus influenzae | TCTCAGTCCCGCACTTICAT |
| 175 | Probe for Klebsiella pneumoniae subsp. | TATTAACCTCATCGCCTTCC |
| | pneumoniae | |
| 176 | Probe for Klebsiella pneumoniae subsp. | GTAACGTCAATCGATGAGGT |
| | pneumoniae | |
| 177 | Probe for Mycoplasma pneumoniae | AAATGGTACAGTCAAACTCTA |
| 178 | Probe for Mycoplasma pneumoniae | CATGCGAACCAAAGTTCTTA |
| 179 | Probe for Pseudomonas aeruginosa | CCGGACGTTATCCCCCACTA |
| 180 | Probe for Pseudomonas aeruginosa | GCTGAATCCAGGAGCAAGCT |
| 181 | Probe for Serratia marcescens | GCGAGTAACGTCAATTGATGA |
| 182 | Probe for Serratia marcescens | CCCCTCTACGAGACTCTAGC |
| 183 | Probe for Serratia marcescens subsp. | GTATTAAGCTCACCACCTTCCT |
| | marcescens | |
| 184 | Probe for Staphylococcus aureus | CTAACATCAGAGAAGCAAGC |
| 185 | Probe for Staphylococcus aureus | AAGACCGTCTTTCACTTTTG |
| 186 | Probe for Staphylococcus aureus | CTTACACATATGTTCTTCCC |
| 187 | Probe for Streptococcus pneumoniae | ATGCAACATCCACTCTTATG |
| 188 | Probe for Streptococcus pneumoniae | CAAGTGCACCTTTTAAGCAA |
| 189 | Probe for Streptococcus pyogenes | CATGCGTTAGTCTCTCTTATGC |
| 190 | Probe for Streptococcus pyogenes | CCATCTCATAGTGGAGCAAT |
| 191 | Probe for Streptococcus pyogenes | CCCACCATCATTCTTCTCTAA |

Hereinafter, the present invention is described in more details in reference to examples but the present invention is not limited to these examples.

### Example 1

In the present example, for multiple bacterial species contained in a sample, an absolute amount of the target nucleic acids (each target nucleic acid) from each bacterium was determined. Specifically, as described below, the creation of a calibration curve, calculation of a relational expression on signal intensities among the probes, and determination of an absolute amount of a target nucleic acid in the sample were carried out.

### 1. Creation of Calibration Curve

A calibration curve was created on a relationship between amounts before amplification and indicators of amounts after amplification of the total of a part of the target nucleic acids and the specific nucleic acid group (nucleic acids for calibration) and a control nucleic acid. Specifically, a calibration curve was created on the relationship between [a ratio of (an indicator of the total amount of a part of the target nucleic acids and the specific nucleic acid group) to (an indicator of an amount of a control nucleic acid after amplification)] and [a ratio of (the total amount of a part of the target nucleic acids and the specific nucleic acid group) to (an amount of the control nucleic acid before amplification)]. The amount of the control nucleic acid before amplification is known, whereby the total amount of all target nucleic acids and the specific nucleic acid group before amplification can be determined based on this calibration curve.

For the creation of the calibration curve, a mixed solution of genome DNA of 11 bacterial species was used as the nucleic acids for calibration. The composition of the mixed solution used is as shown in Table 2. For each genome DNA used, those with ATCC numbers shown in Table 2 were purchased from ATCC (registered trademark) (American Type Culture Collection).

The 16SrRNA gene was used as the target nucleic acids.

TABLE 2

| | | Concentration of |
|---|---|---|
| Strain used | ATCC No. | 16SrRNA gene (pM) |
| Aggregatibacter | 700685D-5 | 11.2 |
| actinomycetemcomitans | | |
| Capnocytophaga gingivalis | 33624D-5 | 9.41 |
| Fusobacterium nucleatum subsp. | 25586D-5 | 12.4 |
| Nucleatum | | |
| Porphyromonas gingivalis | 33277D-5 | 10.0 |
| Prevotella intermedia | 25611D-5 | 7.38 |
| Streptococcus gordonii | 35105D-5 | 10.0 |
| Tannerella forsythia | 43037D-5 | 10.0 |
| Treponema denticola | 35405D-5 | 10.1 |
| Veillonella parvula | 10790D-5 | 9.98 |
| Streptococcus mutans | 700610D-5 | 9.99 |
| Campylobacter rectus | 33238D-5 | 3.32 |
| Total | — | 104 |

The above mixed solution (104 pM) was added to a PCR reaction solution so that the total of concentrations of the 16SrRNA genes in the PCR reaction solution was 0.24 pM and 0.25ⁿ times (n=1, 2, . . . , 9) thereof. Other compositions of the PCR reaction solution are as shown in Table 3. The total solution volume was adjusted to be 20 μL. Two reactions for each condition were carried out.

TABLE 3

| | Final | |
|---|---|---|
| | concentration | |
| Nuclease free water | — | |
| F Primer (for 16SrRNA gene) | 0.20 | μM |
| R Primer (for 16SrRNA gene) | 0.20 | μM |
| F Primer (for control nucleic acid) | 0.20 | μM |
| R Primer (for control nucleic acid) | 0.20 | μM |
| Control nucleic acid | 0.012 | pM |

| | |
|---|---|
| Premix Ex Taq HS | 1x |

The sequences of each of the primers and the control nucleic acid herein are as shown in Tables 4 and 5, respectively, and the F (Forward) primers are Cy5-modified at the 5′-end. R and Y in the sequences shown in Table 4 represent mixed bases wherein R represents A and G and Y represents C and T. Each of the primers are designed in such a way as to bind to a consensus sequence in the 16SrRNA gene of each bacterium. The control nucleic acid is a nucleic acid comprising an artificial sequence consisting of a base sequence required for the amplification and a base sequence of a random combination of bases (Table 5).

TABLE 4

| SEQ | | |
|---|---|---|
| ID | | |
| NO | Name | Sequence |
| 192 | F Primer (for | TACGGGAGGCAGCAG |
| | 16SrRNA gene) | |
| 193 | R Primer (for | CRGGGTATCTAATCCYGTT |
| | 16SrRNA gene) | |
| 194 | F Primer (for | GAGAAGCCTACACAAACGT |
| | control | AACGTC |
| | nucleic acid) | |
| 195 | R Primer (for | CTCTAAAGACCGCTCTATC |
| | control | TCGG |
| | nucleic acid) | |

TABLE 5

| SEQ | | |
|---|---|---|
| ID NO | Name | Sequence |
| 196 | Control | GTGAGAAGCCTACACAAACGTAACGTCAGGGCTAAG |
| | nucleic | ACAAACGCTAACGGTACACCCTAGATGGGAGCTTGT |
| | acid | AGCTAGATCGCTAAGTCCTACCGACATGTAGGCATA |
| | | CTCACGAAGGCAATTCCCTGAAAGCCTCGTCTTATC |
| | | CCGAACTTGGCATCTGCTGATACGTCAGGTTGAACG |
| | | CGTACATTTACCTGTCATGCGTGGGCCTTCTCCGAA |
| | | TAGCCTACGTAGTGATATCGCTGGTCGAATAGGCGG |
| | | ATTGCTCATAAATGCACATTGGCTAAGGCCCACGGA |
| | | ACACGAATCACGTGAGATCACTTACTATTCGACGGA |
| | | ACTACTATACGCACCGGGACATGCAAGTAGCGTCCC |
| | | ACAAGCATAAGGAACTCTATACTCGCCATCTACGCA |
| | | GCTACAGGGGATACACGTATGAGCGGTTACGAAGTA |
| | | AAGCCGAGATAGAGCGGTCTTTAGAG |

PCR enzyme used was Premix Ex Taq (registered trademark) Hot Start Version (TaKaRa), and the reaction was carried out by adjusting 2× concentration including the enzyme and a buffer as provided in such a way as to be 1× concentration.

The PCR reaction was carried out in a thermal cycler under the condition shown in Table 6.

TABLE 6

| 95° C. | 98° C. | 55° C. | 72° C. | 4° C. |
|---|---|---|---|---|
| 1 min | 10 sec | 30 sec | 20 sec | ∞ |

40 Cycles

After the completion of PCR, a hybridization reaction solution (1M Tris-HCl 48 μL, 1M NaCl 48 μL, 0.5% Tween20 20 μL, Nuclease free water 64 μL) was added to the respective reaction solutions (the entire volume of 20 μL). Hybridization was carried out using this solution and a DNA chip in an automated hybridization cleaning apparatus (Mitsubishi Chemical Corporation) at 50° c. for 16 hours. Subsequently, cleaning was carried out.

The DNA chip used in the present example was Genopal (registered trademark), a DNA chip manufactured by Mitsubishi Chemical Corporation. The probe (probe x) loaded on this DNA chip and hybridizable with a specific sequence of each bacterium is the base sequences as set forth in SEQ ID NO: 3 to 18 (Table 7), and for the probe (probe (y)) hybridizable in common with both amplified products of all target nucleic acids and the specific nucleic acid group and for the probe (probe (z)) hybridizable with amplified products of the control nucleic acid, the base sequences as set forth in SEQ ID NO: 2 and 1 were used, respectively. For the detection of nucleic acids from Aggregatibacter actinomycetemcomitans and Prevotella intermedia, a mixture of nucleotides having the base sequences as set forth in SEQ ID NO: 3 and 4 and a mixture of nucleotides having the base sequences as set forth in SEQ ID NO: 9 and 10 were used as the probes, respectively. The 5′-end vinylated oligo-DNA of these sequences were arrayed using Genopal platform (Japanese Patent No. 6299660).

Genopal reader (Mitsubishi Chemical Corporation) was used for detection. Numerical data converted based on the detected fluorescent signals were used for analysis. Those having a condition with the total concentration of the 16SrRNA genes of 0.25ⁿ times (n=8, 9) of 0.24 pM did not change in the signal intensity ratio even with different concentrations and thus were decided as below the lower limit for quantification thereby not used for creating a calibration curve.

The amplified products of the mixed solution of genome DNA of 11 bacterial species and the control nucleic acid contain the amplified products of all of the 16SrRNA genes from the 11 bacterial species (total 16SrRNA genes) and the control nucleic acid.

The signal intensity of the probe (SEQ ID NO: 2) hybridizable with total 16SrRNA genes (the indicator showing the amount of the 16SrRNA genes after amplification) was represented as I_{T}, the signal intensity of the probe (SEQ ID NO: 1) hybridizable with the control nucleic acid was represented as I_{C}, the amount of the 16SrRNA genes before amplification was represented as C_{T}, and the amount of the control nucleic acid before amplification was represented as C_{C}, and these were approximated using [Expression 1: (log₁₀(I_{T}÷I_{C})=a×log₁₀(C_{T}÷C_{C})+b)] (FIG. 1). The slope and intercept of the approximate straight line were determined using LINEST function in a Microsoft software “EXCEL” and the parameters in Expression 1 were determined as a=0.389 and b=0.581.

This calibration curve enabled the determination of the total amount of all target nucleic acids and the specific nucleic acid group before amplification. This “the total amount of all target nucleic acids and the specific nucleic acid group before amplification” was needed when finally determining an absolute amount of the target nucleic acids (each target nucleic acid) from each bacterium.

### 2. Calculation of Relational Expression on Signal Intensities Among Probes

In a later test, for determining an absolute amount of each target nucleic acid based on an occupancy of target nucleic acids (each target nucleic acid) from each bacterium in the “total of all target nucleic acids and the specific nucleic acid group”, the calculation must be carried out by applying the signal intensity of each probe (x) hybridizable with each target nucleic acid and the signal intensity of the probe (y) hybridizable with the “all target nucleic acids and the specific nucleic acid group” to the same expression. On the other hand, as the probe (X) and the probe (y) are likely to be different in the absolute values of the signal intensities, applying both of them to the same expression for the calculation requires to obtain a relational expression on the signal intensities between both of them and convert the signal intensity of the probe (x) to a value equivalent to the signal intensity of the probe (y).

For this reason, in the present example, relational expressions between the signal intensities of probes (probe (x)) (SEQ ID NO: 3 to 18) hybridizable respectively with specific sequences of the 14 bacterial species (Table 7) and the signal intensity of the probe (probe (y)) (SEQ ID NO: 2) hybridizable with all of the 16SrRNA genes (total 16SrRNA genes) from the 14 bacterial species were calculated by the bacterial species.

For each genome DNA used, those with the numbers shown in Table 7 were purchased from ATCC (registered trademark) (American Type Culture Collection) or DSMZ (Deutsche Sammlung Von Mikroorganismen Und Zellkulturen).

TABLE 7

| SEQ ID NO | | |
|---|---|---|
| of probe | Name of bacteria | Number |
| 3, 4 | Aggregatibacter | ATCC 700685D-5 |
| | actinomycetemcomitans | |
| 5 | Campylobacter rectus | ATCC 33238D-5 |
| 6 | Capnocytophaga gingivalis | ATCC 33624D-5 |
| 7 | Fusobacterium nucleatum | ATCC 25586D-5 |
| 8 | Porphyromonas gingivalis | ATCC 33277D-5 |
| 9, 10 | Prevotella intermedia | ATCC 25611D-5 |
| 11 | Prevotella nigrescens | DSMZ NO. 13386 |
| 12 | Selenomonas noxia | DSMZ NO. 19578 |
| 13 | Streptococcus constellatus | DSMZ NO. 25819 |
| 14 | Streptococcus gordonii | ATCC 35105D-5 |
| 15 | Streptococcus intermedius | DSMZ NO. 20573 |
| 16 | Tannerella forsythia | ATCC 43037D-5 |
| 17 | Treponema denticola | ATCC 35405D-5 |
| 18 | Veillonella parvula | ATCC 10790D-5 |

The following experiment was carried out for each genome DNA.

The PCR reaction solution was first prepared in such a way that weights of the genome DNA contained in the solution were 1 pg, 0.5 pg, and 0.1 g. Other compositions and the solution volume were the same as in the above “1. Creation of calibration curve.”

The PCR reaction, hybridization and detection were carried out in the same manner as in the above “1. Creation of calibration curve” and numerical data converted based on the detected fluorescent signals were used for analysis. Specifically, DNA of all 16SrRNA genes from the above 14 bacterial species were amplified by PCR reaction and hybridized with the probes loaded on the DNA chip. The signal intensity of each probe (probe (x)) hybridizable respectively with specific base sequence of each bacterium was represented as I_{US} and the signal intensity of the probe (probe (y)) hybridizable with total 16SrRNA genes was represented as I_{TS}, and these were approximated using [Expression 5: I_{TS}=E_{S}×I_{US}] for each of the bacterial species. This relational expression, that is, the relational expression between the signal intensity of each probe (x) and the signal intensity of the probe (y), was determined for all of the 14 bacterial species.

The results on the genome DNA of Aggregatibacter actinomycetemcomitans and Campylobacter rectus are shown as representative in FIG. 2. As a result of determining the slope of the approximate straight line using an option to force an intercept of 0 with LINEST function in a Microsoft software “EXCEL”, the parameter E_{S} in Expression 5 was determined as shown in Table 8.

TABLE 8

| Probe No. | Name of bacteria | Eₛ |
|---|---|---|
| 3, 4 | Aggregatibacter | 0.526 |
| | actinomycetemcomitans | |
| 5 | Campylobacter rectus | 0.708 |
| 6 | Capnocytophaga gingivalis | 0.447 |
| 7 | Fusobacterium nucleatum | 0.572 |
| 8 | Porphyromonas gingivalis | 1.15 |
| 9, 10 | Prevotella intermedia | 0.804 |
| 11 | Prevotella nigrescens | 2.62 |
| 12 | Selenomonas noxia | 0.925 |
| 13 | Streptococcus constellatus | 2.78 |
| 14 | Streptococcus gordonii | 0.488 |
| 15 | Streptococcus intermedius | 0.427 |
| 16 | Tannerella forsythensis | 0.258 |
| 17 | Treponema denticola | 0.175 |
| 18 | Veillonella parvula | 2.95 |

### 3. Determination of Absolute Amounts of Target Nucleic Acids in a Sample

A saliva evaluation test was carried out with the cooperation of 1 healthy adult (subject).

Saliva was collected at the time of waking up, crushed with bead, treated with proteinase K (QUIAGEN), and extracted DNA using QIAamp 96 DNA Blood Kit (QIAGEN). DNA extracted from the saliva contained genome DNA of the above 14 bacterial species to be measured, genome DNA of the bacterial species other than the bacterial species to be measured, and other DNA (FIG. 4A).

A concentration of the extracted DNA was measured using NanoDrop (trademark) (Thermo Fisher Scientific) and a PCR reaction solution was prepared based on the measured concentration in such a way that a weight of the extracted DNA contained in the solution was 100 pg. Other composition and the solution volume were the same as in the above “1. Creation of calibration curve.”

The PCR reaction, hybridization and detection were carried out in the same manner as in the above “1. Creation of calibration curve” and numerical data converted based on the detected fluorescent signals were used for analysis.

The amplified products of the DNA extracted from the saliva sample and the control nucleic acid contained (i) the 16SrRNA genes (target nucleic acids) from the above 14 bacterial species to be measured, (ii) the 16SrRNA genes from bacterial species other than the bacterial species to be measured, which are the specific nucleic acid group to be collectively amplified with all target nucleic acids by amplification (specific nucleic acid group), and (iii) the amplified control nucleic acid (FIG. 4B).

The total of the above (i) and (ii) is applicable to the above “the total of all target nucleic acids and the specific nucleic acid group” or “total 16SrRNA genes.”

An amount of substance (mol) C_{TM} of total 16SrRNA genes added to the PCR reaction solution was calculated using the signal intensities I_{TM} of probes of total 16SrRNA genes and the parameters determined in “1. Creation of calibration curve” in accordance with Expression 3 [C_{TM}=C_{C}×10\hat{ }((log₁₀(I_{TM}÷I_{CM})−0.581)÷0.389)] (FIG. 4C) and the number of copies was calculated by multiplying C_{TM} by Avogadro's number. The number of copies of total 16SrRNA gene in 1 mL of the saliva was calculated based on the dilution ratios at the extraction step and the PCR reaction solution preparation step.

Subsequently, the number of 16SrRNA gene copies of each bacterium was calculated from the signal intensity I_{US} of each probe (x) specifically hybridizable with each bacterium, the parameters determined in “2. Calculation of relational expression on signal intensities among probes” and the number of copies of total 16SrRNA genes per mL of the above saliva in accordance with Expression 6 (I_{TSM}=E_{S}×I_{USM}) and Expression 7 (R_{S}=I_{TSM}÷I_{TM}) (FIG. 4D).

The calculated number of copies of each 16SrRNA gene per mL of the saliva are as shown in Table 9.

TABLE 9

| SEQ ID NO | | Number of |
|---|---|---|
| of probe | Name | copies |
| 2 | Total 16SrRNA genes | 4.3.E+07 |
| 3, 4 | Aggregatibacter | 4.6.E+04 |
| | actinomycetemcomitans | |
| 5 | Campylobacter rectus | 7.4.E+04 |
| 6 | Capnocytophaga gingivalis | 6.4.E+04 |
| 7 | Fusobacterium nucleatum | 5.0.E+05 |
| 8 | Porphyromonas gingivalis | 1.4.E+05 |
| 9, 10 | Prevotella intermedia | 7.1.E+04 |
| 11 | Prevotella nigrescens | 1.1.E+05 |
| 12 | Selenomonas noxia | 2.2.E+04 |
| 13 | Streptococcus constellatus | 1.9.E+04 |
| 14 | Streptococcus gordonii | 2.8.E+05 |
| 15 | Streptococcus intermedius | 1.4.E+05 |
| 16 | Tannerella forsythia | 1.6.E+04 |
| 17 | Treponema denticola | 4.1.E+04 |
| 18 | Veillonella parvula | 3.1.E+05 |

### Example 2

In the present example, a calibration curve was created under different conditions from Example 1.

The composition of a mixed solution used as the nucleic acids for calibration was the same as in Example 1.

The above mixed solution was added to a PCR reaction solution so that the total of concentrations of the 16SrRNA genes in the PCR reaction solution was 24 pM and ( 1/16)ⁿ times (n=1, 2, . . . , 5) thereof. Other compositions of the PCR reaction solution are as shown in Table 10. The total solution volume was adjusted to be 20 μL. One reaction was carried out for each condition.

TABLE 10

| | Final concentration | |
|---|---|---|
| Nuclease free water | — | |
| F Primer (for 16SrRNA gene) | 1.0 | μM |
| R Primer | 1.0 | μM |
| Control nucleic acid | 0.025 | pM |

| | |
|---|---|
| Premix Ex Taq HS | 1x |

The sequences of each of the primers and the control nucleic acid herein are as shown in Tables 11 and 12, respectively, and the F primers are Cy5-modified at the 5′-end. “I” in the sequence of Table 11 represents inosine.

TABLE 11

| SEQ ID | | |
|---|---|---|
| NO | Name | Sequence |
| 192 | F Primer (for | TACGGGAGGCAGCAG |
| | 16SrRNA gene) | |
| 197 | R Primer | TACCTGGGTATCTAATCC |

TABLE 12

| SEQ | | |
|---|---|---|
| ID | | |
| NO | Name | Sequence |
| 198 | nucleic | GTTCCTACGGGAGGCAGCAGTAGGGCTAAGACAAAC |
| | acid | GCTAACGGTACACCCTAGATGGGAGCTTGTAGCTAG |
| | Control | ATCGCTAAGTCCTACCGACATGTAGGCATACTCACG |
| | | AAGGCAATTCCCTGAAAGCCTCGTCTTATCCCGAAC |
| | | TTGGCATCTGCTGATACGTCAGGTTGAACGCGTACA |
| | | TTTACCTGTCATGCGTGGGCCTTCTCCGAATAGCCT |
| | | ACGTAGTGATATCGCTGGTCGAATAGGCGGATTGCT |
| | | CATAAATGCACATTGGCTAAGGCCCACGGAACACGA |
| | | ATCACGTGAGATCACTTACTATTCGACGGAACTACT |
| | | ATACGCACCGGGACATGCAAGTAGCGTCCCACAAGC |
| | | ATAAGGAACTCTATACTCGCCATCTACGCAGCTACA |
| | | GGGGATACACGTATGAGCGGTTACGAAGTAAAGGGT |
| | | AGCAAACAGGATTAGATACCCTGAAA |

The PCR reaction was carried out in a thermal cycler under the condition shown in Table 13.

TABLE 13

| 95° C. | 98° C. | 55° C. | 72° C. | 4° C. |
|---|---|---|---|---|
| 1 min | 10 sec | 30 sec | 20 sec | ∞ |

25 Cycles

The hybridization and detection were carried out in the same manner as in “1. Creation of calibration curve” of Example 1 and numerical data converted based on the detected fluorescent signals were used for analysis.

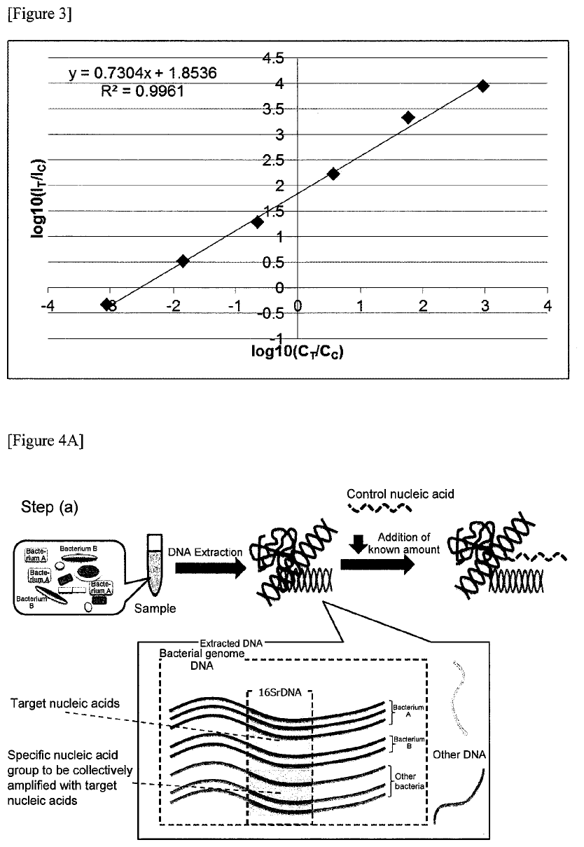

The signal intensity of the probe (SEQ ID NO: 2) hybridizable with total 16SrRNA genes is represented as I_{T}, the signal intensity of the probe (SEQ ID NO: 1) hybridizable with the control nucleic acid was represented as I_{C}, the amount of the 16SrRNA genes before amplification was represented as C_{T}, and the amount of the control nucleic acid before amplification was represented as C_{C}, and these were approximated by [Expression 1: (log 10(I_{T}÷I_{C})=a×log 10(C_{T}÷C_{C})+b)] (FIG. 3). The slope and intercept of the approximate straight line were determined using LINEST function in a Microsoft software “EXCEL” and the parameters in Expression 1 were determined as a=0.730 and b=1.85.

Examples 1 and 2 showed that the method of the present invention can determine an absolute amount of multiple types of target nucleic acids contained in a sample simultaneously and in a simple manner. Additionally, the method of the present invention showed that only a single type of a nucleic acid needs to be used as the control nucleic acid and a plural type of control nucleic acids do not need to be used.

### Example 3

In the present example, for bacteria contained in a sample, an absolute amount of target nucleic acids (each target nucleic acid) from each bacterium was determined under different conditions from Example 1. Standard samples having a known composition were used as a sample, and measured values were compared with theoretical values.

### 1. Creation of Calibration Curve

As nucleic acids for calibration for creating a calibration curve, the total of three types of solutions, commercial 20 Strain Even Mix Genomic Material (ATCC (registered trademark) MSA-1002™), 20 Strain Staggerd Mix Genomic Material (ATCC (registered trademark) MSA-1003™) and, in addition, a mixed solution of genome DNA of seven bacterial species having the composition shown in Table 14 were used as the standard samples.

TABLE 14

| | | Concentration of |
|---|---|---|
| Strain used | ATCC No. | 16SrRNA gene (pM) |
| Aggregatibacter | 700685D-5 | 14.3 |
| actinomycetemcomitans | | |
| Fusobacterium nucleatum | 25586D-5 | 14.3 |
| subsp. nucleatum | | |
| Porphyromonas gingivalis | 33277D-5 | 13.6 |
| Prevotella intermedia | 25611D-5 | 14.3 |
| Streptococcus gordonii | 35105D-5 | 14.3 |
| Streptococcus mutans | 700610D-5 | 14.3 |
| Veillonella parvula | 10790D-5 | 14.3 |
| Total | — | 99.4 |

The above standard samples were subjected to PCR reaction under the conditions (template concentration, repeat count (N count)) shown in Table 15 and the obtained PCR products were measured using the DNA chip. The composition of the PCR reaction solution other than the nucleic acids for calibration was as shown in Table 16.

TABLE 15

| Nucleic acids | Concentration of | Repeat count |
|---|---|---|
| for calibration | 16SrRNA gene (pM) | (N count) |
| MSA1003 | 9.39 × 0.25\hat{ }n | 3 Each |
| Staggerd Mix | (n = 1, 2 . . . 9) | |
| | 8.79 | 3 |
| | 9.50 | 2 |
| MSA1002 | 2.43 | 3 |
| Even Mix | 2.79 | 2 |
| 0C93 MIX | 24.8 × 0.0625\hat{ }n | 2 Each (n = 1, 4) or |
| | (n = 1, 2, . . . , 6) | 4 each (n = 2, 3, 5, 6) |

TABLE 16

| Component | Final concentration | |
|---|---|---|
| Nuclease free water | — | |
| F Primer (for 16SrRNA gene) | 1.0 | μM |
| R Primer | 1.0 | μM |
| Control nucleic acid | 0.025 | pM |

| | |
|---|---|
| Premix Ex Taq HS | 1x |

The primers used are the same as in Example 2 and the base sequence of the control nucleic acid is as shown in Table 17.

TABLE 17

| SEQ | | |
|---|---|---|
| ID | | |
| NO | Name | Sequence |
| 199 | Control | GTTCCTACGGGAGGCAGCAGTAGGGCTAAGAC |
| | nucleic | AAACGCTAACGGTACACCCTAGATGGGAGCTT |
| | acid | GTAGCTAGATCGCTAAGTCCTACCGACATGTA |
| | | GGCATACTCACGAAGGCAATTCCCTGAAAGCC |
| | | TCGTCTTATCCCGAACTTGGCATCTGCTGATA |
| | | CGTCAGGITGAACGCGTACATTTACCTGTCAT |
| | | GCGTGGGCCTTCTCCGAATAGCCTACGTAGTG |
| | | ATATCGCTGGTCGAATAGGCGGATTGCTCATA |
| | | AATGCACATTGGCTAAGGCCCACGGAACACGA |
| | | ATCACGTGAGATCACTTACTATTCGACGGAAC |
| | | TACTATACGCACCGGGACATGCAAGTAGCGTC |
| | | CCACAAGCATAAGGAACTCTATACTCGCCATC |
| | | TACGCAGCTACAGGGGATACACGTATGAGCGG |
| | | TTACGAAGTAAAGGGTAGCAACAGGATTAGAT |
| | | ACCCTGGTAGTCCAC |

The PCR reaction was carried out in a thermal cycler under the same conditions as in “1. Creation of calibration curve” of Example 1. The DNA chip used in the present example has the same structure as in Example 1 but a new DNA chip on which the probe for Actinomyces odontolyticus (SEQ ID NO: 19) and the probe for Streptococcus nutans (SEQ ID NO: 38) were additionally loaded in addition to the probe (probe (x)) hybridizable with the specific sequence of each bacterium was prepared.

The hybridization and detection were carried out in the same manner as in “1. Creation of calibration curve” of Example 1 and numerical data converted based on the detected fluorescent signals were used for analysis.

The signal intensity of the probe (SEQ ID NO: 2) hybridizable with total 16SrRNA genes was represented as I_{T}, the signal intensity of the probe (SEQ ID NO: 1) hybridizable with the control nucleic acid was represented as I_{C}, the amount of the 16SrRNA genes before amplification was represented as C_{T}, and the amount of the control nucleic acid before amplification was represented as C_{C}, and these were approximated using [Expression 2: log 10(I_{T}÷I_{C})=b×(log 10(C_{T}÷C_{C})−c)÷(a−|log 10(C_{T}÷C_{C})−c|)] (FIG. 5). An approximate expression was created using the function called “SOLVER” in a Microsoft software “EXCEL” and the parameters in Expression 2 were determined as a=8.07, b=3.43, and c=0.119. Additionally, the signal intensities obtained were confirmed to have been plotted on the substantially same curved line regardless the type of nucleic acids for calibration used.

### 2. Calculation of Relational Expression on Signal Intensities Among Probes

A relational expression between the signal intensities of the probes (probe (x)) (SEQ ID NO: 8, 19, 38) hybridizable respectively with specific base sequences of three bacterial species (Table 18) and the signal intensity of the probe (probe (y)) (SEQ ID NO: 2) hybridizable with all of the 16SrRNA genes (total 16SrRNA genes) from the three bacterial species was herein calculated by the bacterial species.

The DNA used are synthetic nucleic acids of the base sequences described in Table 18.

TABLE 18

| SEQ ID NO | | Base sequence of synthetic |
|---|---|---|
| of probe | Name of bacteria | nucleic acid used |
| 8 | Porphyromonas | Sequence of ID: AB035459.1 of |
| | gingivalis | GenBank |
| 19 | Actinomyces | Sequence from 1281646^{th} base to |
| | odontolyticus | 1499^{th} base of sequence of ID: |
| | | AAYI02000004.1 of GenBank |
| 38 | Streptococcus mutans | Sequence of ID: AY188348 of |
| | | GenBank |

The following experiment was carried out for each DNA of the three bacterial species.

The PCR reaction solution was first prepared in such a way that amounts of substance of DNA contained in the solution were 0.5 amol, 0.05 amol, and 0.005 amol. Other compositions and the solution volume are the same as in the above “1. Creation of calibration curve” of the present example.

The PCR reaction, hybridization and detection were carried out in the same manner as in the above “1. Creation of calibration curve” of the present example and numerical data converted based on the detected fluorescent signals were used for analysis.

Specifically, the above three types of DNA were amplified by PCR reaction and hybridized with the probes loaded on the DNA chip. The signal intensity of each probe (probe (x)) hybridizable respectively with specific base sequence of each bacterium was represented as I_{US} and the signal intensity of the probe (probe (y)) hybridizable with total 16SrRNA genes was represented as I_{TS}, and these were approximated using [Expression 5: I_{TS}=E_{S}×I_{US}] by the bacterial species. The relational expression, that is, the relational expression between the signal intensity of each probe (x) and the signal intensity of the probe (y) was determined for all three bacterial species.

The results on the three types of DNA are shown in FIG. 6. As a result of determining the slope of the approximate straight line using an option to force an intercept of 0 with LINEST function in a Microsoft software “EXCEL”, the parameter E_{S} in Expression 5 was determined as shown in Table 19.

TABLE 19

| Name of bacteria | Eₛ |
|---|---|
| Porphyromonas gingivalis | 1.45 |
| Actinomyces odontolyticus | 3.30 |
| Streptococcus mutans | 1.15 |

### 3. Determination of Absolute Amounts of Target Nucleic Acids in a Sample

Quantitativity was verified using 20 Strain Even Mix Genomic Material (ATCC (registered trademark) MSA-1002™) as a sample having a known concentration. The number of copies of genome DNA of the 3 bacteria shown in the above Table 18 contained in this sample was already known to be 5% of the total, respectively. That is, a theoretical value of the number of copies of genome DNA of each bacterium per weight of this sample can be calculated based on this composition information of the genome DNA and the information for the number of copies of 16SrRNA in 1 genome of each bacterial genome DNA and genome size.

The PCR reaction solution was prepared in such a way that a weight of the sample DNA contained in the solution was 211 pg. Other compositions and the solution volume were the same as in the above “1. Creation of calibration curve.”

The PCR reaction, hybridization and detection were carried out in the same manner as in the above “1. Creation of calibration curve” and numerical data converted based on the detected fluorescent signals were used for analysis.

An amount of substance (mol) C_{TM} of total 16SrRNA genes added to the PCR reaction solution was calculated using the signal intensities I_{TM} of probes of total 16SrRNA genes and the parameters determined in “1. Creation of calibration curve” in accordance with Expression 4 [C_{TM}=C_{C}×10\hat{ }(8.43×log 10(I_{TM}÷I_{CM})÷(3.43+|log 10(I_{TM}÷I_{CM})|)+0.119)] (FIG. 4C) and the number of copies was calculated by multiplying C_{TM} by Avogadro's number.

Subsequently, the number of copies of 16SrRNA gene of each bacterium was calculated from the signal intensity of each probe (x) specifically hybridizable with each bacterium I_{US}, the parameters determined in “2. Calculation of relational expression on signal intensities among probes” and the number of total 16SrRNA gene copies calculated in the above in accordance with Expression 6 (I_{TSM}=E_{S}×I_{USM}) and Expression 7 (R_{S}=I_{TSM}÷I_{TM}) (FIG. 4D).

Further, the number of copies of genome DNA of each bacterium was calculated based on the number of copies of 16SrRNA gene of the 3 bacteria calculated and the number of copies of 16SrRNA in 1 genome of respective bacteria (see annotation information on sequences respectively with Genbank ID of AP009380.1, NZ_DS264586.1, and AE014133.2). The number of copies of total genome DNA was calculated using 5.25, which was a weighted average value of the bacterial composition of 20 Strain Even Mix Genomic Material (ATCC (registered trademark) MSA-1002™) used as the sample.

The measured values of the number of copies of each genome DNA added to the PCR reaction solution and theoretical values are shown in Table 20. The differences between the measured values and the theoretical values are matched with each other within an error range of about 2 times, with the exception of Actinomyces odontolyticus.

TABLE 20

| | Number of copies | Number of copies | Measured value/ |
|---|---|---|---|
| Name of bacteria | (measured value) | (theoretical value) | theoretical value |
| Total genome DNA | 8.65.E+04 | 5.74.E+04 | 1.51 |
| Porphyromonas gingivalis | 4.96.E+03 | 2.87.E+03 | 1.73 |
| Actinomyces odontolyticus | 5.55.E+02 | 2.87.E+03 | 0.193 |
| Streptococcus mutans | 6.69.E+03 | 2.87.E+03 | 2.33 |

The above results showed that the method of the present invention can determine an absolute amount of two or more types of target nucleic acids in a sample with a high accuracy.

### Example 4

In the present example, λ exonuclease treatment was added after the PCR amplification to determine an absolute amount, which was compared with theoretical values.

### 1. Creation of Calibration Curve

For creation of a calibration curve, a commercial 20 Strain Even Mix Genomic Material (ATCC (registered trademark) MSA-1002™) was used as the nucleic acids for calibration. The above solution was added to a PCR reaction solution so that the total of concentrations of the 16SrRNA genes in the PCR reaction solution was 1.60 pM and 0.25n times (n=1, 2, . . . , 8) thereof. Other compositions of the PCR reaction solution are the same as in Example 3. The total solution volume was adjusted to be 20 μL. One reaction for each condition was carried out.

The F primers and the control nucleic acid used herein are the same as in Example 3. On the other hand, the R primers used had the same sequence as in Example 3 and were phosphorylated at the 5′-end.

The PCR reaction was carried out in the same manner as in Example 3, and subsequently the 2 exonuclease-treated reaction solution was prepared to have a total solution volume of 50 μL using the entire volume of the PCR reaction solution with the composition shown in Table 21. The λ exonuclease-treated reaction was carried out in a thermal cycler under the conditions shown in Table 22. The λ exonuclease and the buffer used are manufactured by NEW ENGLAND BioLabs, Inc.

TABLE 21

| Component | Final concentration |
|---|---|
| Nuclease free water | — |
| PCR Reaction solution | — |
| Lambda Exonuclease Reaction | 1x |
| Buffer | |
| λ Exonuclease | 0.3 Unit/μl |

TABLE 22

| 37° C. | 75° C. | 4° C. |
|---|---|---|
| 30 min | 10 min | ∞ |

After the completion of reaction by λ exonuclease, a hybridization reaction solution (1M Tris-HCl 48 μL, 1M NaCl 48 μL, 0.5% Tween20 20 μL, Nuclease free water 34 μL) was added to the respective reaction solutions (the entire volume of 50 μL). Hybridization was carried out using this solution and a DNA chip in an automated hybridization cleaning apparatus (Mitsubishi Chemical Corporation) at 50° C. for 16 hours. Subsequently, cleaning was carried out. The DNA chip used was the same as in Example 3.

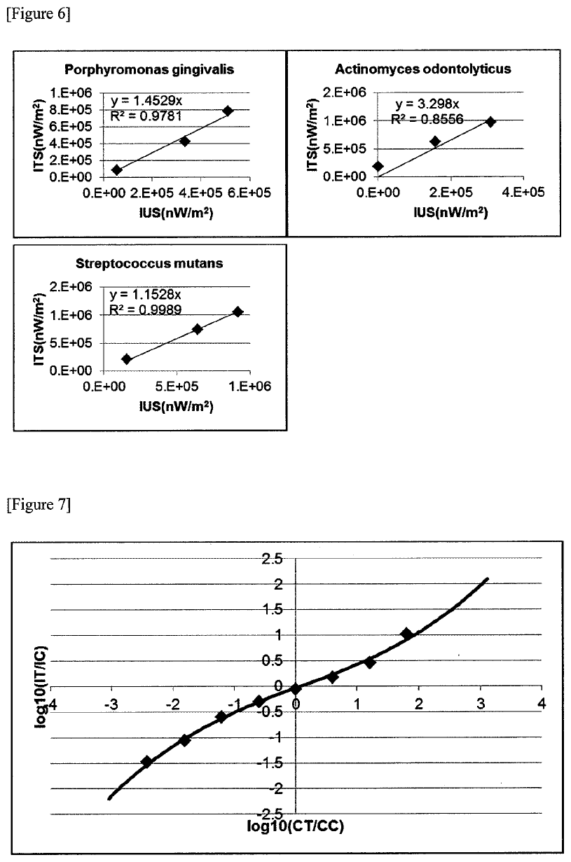

The detection was carried out in the same manner as in “1. Creation of calibration curve” of Example 3 and numerical data converted based on the detected fluorescent signals were used for analysis. The approximation using Expression 2 was also carried out in the same manner as in “1. Creation of calibration curve” of Example 3 (FIG. 7) and the parameters were determined as a=7.29, b=2.94, and c=0.0746.

### 2. Calculation of Relational Expression on Signal Intensities Among Probes

A relational expression between the signal intensities of the probes (probe (x)) (SEQ ID NO: 8, 19, 38) hybridizable respectively with specific sequences of three bacterial species (Table 18) quantified in Example 3 and the signal intensity of the probe (probe (y)) (SEQ ID NO: 2) hybridizable with all of the 16SrRNA genes (total 16SrRNA genes) from the three bacterial species was herein calculated by the bacterial species. That is, the exonuclease treatment after the PCR reaction and the addition of the hybridization reaction solution were carried out in the same manner as in the above “1. Creation of calibration curve” and others were carried out in the same manner as in “2. Calculation of relational expression on signal intensities among probes” of Example 3 to determine the relational expression between the signal intensity of each probe (x) and the signal intensity of the probe (y) for all of the three bacterial species.

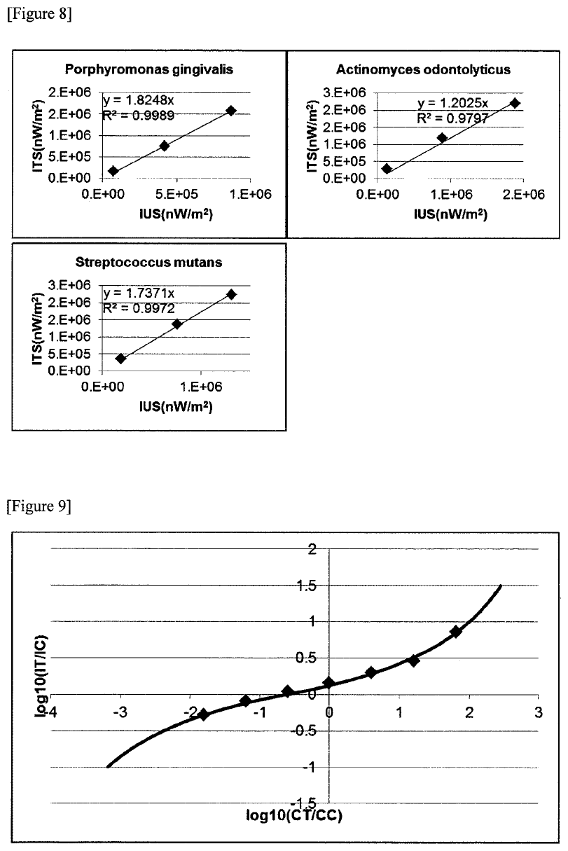

The results on the DNA of the three bacterial species are shown in FIG. 8. The parameter E_{S} in Expression 5 was determined as shown in Table 23.

TABLE 23

| SEQ ID NO | | |
|---|---|---|
| of probe | Name of bacteria | Eₛ |
| 8 | Porphyromonas gingivalis | 1.82 |
| 19 | Actinomyces odontolyticus | 1.20 |
| 38 | Streptococcus mutans | 1.74 |

### 3. Determination of Absolute Amounts of Target Nucleic Acids in a Sample

A quantification experiment was carried out using a commercial 20 Strain Even Mix Genomic Material (ATCC (registered trademark) MSA-1002™) as the standard sample.

The λ exonuclease treatment after the PCR reaction and the addition of the hybridization reaction solution were carried out in the same manner as in the above “1. Creation of calibration curve” and others were carried out in the same manner as in “3. Determination of absolute amounts of target nucleic acids in a sample” of Example 3 to calculate the number of copies of genome DNA of each bacterium for all of the three bacterial species.

The measured values of the calculated number of copies of each genome DNA in the PCR reaction solution and theoretical values are as shown in Table 24.

TABLE 24

| | Number of copies | Number of copies | Measured value/ |
|---|---|---|---|
| Name of bacteria | (measured value) | (theoretical value) | theoretical value |
| All genome DNA | 5.02.E+04 | 5.74.E+04 | 0.874 |
| Porphyromonas gingivalis | 2.56.E+03 | 2.87.E+03 | 0.892 |
| Actinomyces odontolyticus | 2.34.E+03 | 2.87.E+03 | 0.814 |
| Streptococcus mutans | 5.75.E+03 | 2.87.E+03 | 2.00 |

In the above results, the smaller the difference between the measured value and the theoretical value in the number of copies (measured value/theoretical value is close to 1), the higher the accuracy of the absolute amount measurement is shown.

The above results showed that the method of the present invention comprising the step of enriching nucleic acids after amplification for a single chain (a single strand) was able to determine absolute amounts of two or more types of target nucleic acids in a sample with a further higher accuracy than the results of Example 3 (Table 20). Particularly, in the determination of absolute amounts of the target nucleic acids of Actinomyces odontolyticus in Example 4, the notable enhancement in the measurement accuracy of absolute amounts was recognized when compared with the results of Example 3 (Table 20).

### Example 5

In the present example, a part of the PCR reaction conditions was altered and the comparison with the theoretical value was carried out in the same manner as in Example 3.

### 1. Creation of Calibration Curve

The nucleic acids for calibration used for creating a calibration curve, the composition of the PCR reaction solution, the primers and the control nucleic acid used are the same as in Example 4.

The PCR reaction was carried out in a thermal cycler under the condition shown in Table 25, subsequently the hybridization and detection were carried out in the same manner as in “1. Creation of calibration curve” of Example 1 and numerical data converted based on the detected fluorescent signals were used for analysis. The DNA chip used was the same as in Example 3.

TABLE 25

| 95° C. | 98° C. | 55° C. | 72° C. | 72° C. | 4° C. |
|---|---|---|---|---|---|
| 1 min | 10 sec | 30 sec | 1 min | 10 min | ∞ |

40 Cyclles

The approximation using Expression 2 was carried out in the same manner as in “1. Creation of calibration curve” of Example 3 (FIG. 9) and the parameters were determined as a=4.74, b=0.834, and c=−0.593.

### 2. Calculation of Relational Expression on Signal Intensities Among Probes

A relational expression between the signal intensities of the probes (probe (x)) (SEQ ID NO: 19 and 38) hybridizable respectively with specific sequences of two bacterial species, Actinomyces odontolyticus and Streptococcus mutans, of the three bacterial species quantified in Example 3 (Table 18) and the signal intensity of the probe (probe (y)) (SEQ ID NO: 2) hybridizable with all of the 16SrRNA genes (total 16SrRNA genes) from the two bacterial species was herein calculated by the bacterial species. That is, the PCR reaction was carried out in the same manner as in the above “1. Creation of calibration curve” and others were carried out in the same manner as in “2. Calculation of relational expression on signal intensities among probes” of Example 3 to determine the relational expression between the signal intensity of each probe (x) and the signal intensity of the probe (y) for the two bacterial species.

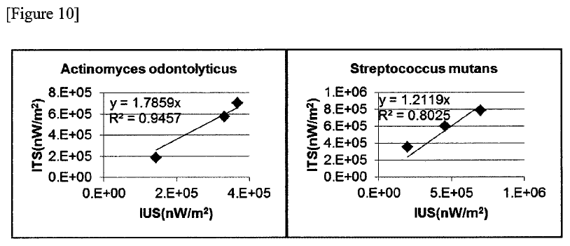

The results of two types of DNA are shown in FIG. 10. The parameter E_{S} in Expression 5 was determined as shown in Table 26.

TABLE 26

| SEQ ID NO | | |
|---|---|---|
| of probe | Name of bacteria | Eₛ |
| 19 | Actinomyces odontolyticus | 1.79 |
| 38 | Streptococcus mutans | 1.21 |

### 3. Determination of Absolute Amounts of Target Nucleic Acids in a Sample

A quantification experiment was carried out using a commercial 20 Strain Even Mix Genomic Material (ATCC (registered trademark) MSA-1002™) as the standard sample.

The PCR reaction was carried out in the same manner as in the above “1. Creation of calibration curve” and others were carried out in the same manner as in “3. Determination of absolute amounts of target nucleic acids in a sample” of Example 3 to calculate the number of copies of genome DNA of each bacterium for the two bacterial species.

The measured values of the calculated number of copies of each genome DNA in the PCR reaction solution and theoretical values are as shown in Table 27.

TABLE 27

| | Number of copies | Number of copies | Measured value/ |
|---|---|---|---|
| Name of bacteria | (measured value) | (theoretical value) | theoretical value |
| Total genome DNA | 8.74.E+04 | 5.74.E+04 | 1.52 |
| Actinomyces odontolyticus | 2.83.E+03 | 2.87.E+03 | 0.986 |
| Streptococcus mutans | 4.42.E+03 | 2.87.E+03 | 1.54 |

The above results showed that the method of the present invention was able to determine absolute amounts of two or more types of target nucleic acids in a sample with a further higher accuracy than the results of Example 3 (Table 20). Particularly, in the determination of absolute amounts of the target nucleic acids of Actinomyces odontolyticus in Example 5, the significant enhancement in the measurement accuracy of absolute amounts was recognized when compared with the results of Example 3 (Table 20).

### INDUSTRIAL APPLICABILITY

According to the present invention, an absolute quantification of multiple types of target nucleic acids respectively (each target nucleic acid) is enabled in a simple manner.

### SEQ ID NOS: 1 to 199: Synthetic DNA

1. A method for determining absolute amounts of two or more types of target nucleic acids in a sample, the method comprising:
(a) mixing a sample with a known amount of a control nucleic acid;
(b) co-amplifying all target nucleic acids and a specific nucleic acid group to be collectively amplified with the all target nucleic acids in the sample and the control nucleic acid, thereby generating amplified all target nucleic acids, an amplified specific nucleic acid group and an amplified control nucleic acid,
(c) determining a total amount of the all target nucleic acids and the specific nucleic acid group in the sample based on an indicator of a total amount of the amplified all target nucleic acids and the amplified specific nucleic acid group and an indicator of an amount of the amplified control nucleic acid; and
(d) calculating an absolute amount of each target nucleic acid in the sample based on an occupancy of each target nucleic acid in a total of the all target nucleic acids and the specific nucleic acid group which is calculated from the indicator of the total amount of the amplified all target nucleic acids and the amplified specific nucleic acid group and an indicator of an amount of each amplified target nucleic acid.

2. The method according to claim 1, wherein the control nucleic acid is an artificial sequence consisting of a base sequence required for amplification and a random base sequence in which a part or all of bases thereof are in a random combination.

3. The method according to claim 1, wherein the co-amplification is carried out by a technique selected from the group consisting of Polymerase Chain Reaction (PCR) method, Loop-Mediated Isothermal Amplification (LAMP) method, and in vitro transcription.

4. The method according to claim 3, wherein the co-amplification is carried out by PCR method.

5. The method according to claim 1, wherein the sample is at least one selected from the group consisting of a food, a drink, a biological sample, an environmental sample, and a sample from industrial process.

6. The method according to claim 5, wherein the sample is at least one selected from the group consisting of an activated sludge, a soil, a river water, a seawater, a hot spring water, a drinking water, a processed food, a fermenter culture, and a tissue, a cell and a body fluid collected from at least one eukaryote.

7. The method according to claim 5, wherein the biological sample is present and is at least one selected from the group consisting of saliva, plaque, gingival crevicular fluid (GCF), feces, and a skin-derived sample collected from at least one mammal.

8. The method according to claim 1, wherein the target nucleic acids are bacterial ribosomal RNA (rRNA) genes.

9. The method according to claim 8, wherein the rRNA genes are rRNA genes of at least two species of bacteria selected from the group consisting of bacteria belonging to the Abiotrophia genus, Achromobacter genus, Acinetobacter genus, Actinomyces genus, Aerococcus genus, Aggregatibacter genus, Alloprevotella genus, Alloscardovia genus, Anaerococcus genus, Anaeroglobus genus, Arcanobacterium genus, Atopobium genus, Bacillus genus, Bacteroides genus, Bifidobacterium genus, Bordetella genus, Brevundimonas genus, Brucella genus, Burkholderia genus, Campylobacter genus, Candidatus Absconditabacteria (SR1), Candidatus Saccharibacteria (TM7), Capnocytophaga genus, Cardiobacterium genus, Catonella genus, Chlamydia genus, Chlamydophila genus, Chryseobacterium genus, Citrobacter genus, Clostridium genus, Collinsella genus, Corynebacterium genus, Coxiella genus, Cronobacter genus, Cryptobacterium genus, Curvibacter genus, Dialister genus, Eggerthella genus, Eikenella genus, Elizabethkingia, Enterobacter genus, Enterococcus genus, Escherichia genus, Eubacterium genus, Filifactor genus, Finegoldia genus, Fusobacterium genus, Gardnerella genus, Gemella genus, Granulicatella genus, Haemophilus genus, Helicobacter genus, Kingella genus, Klebsiella genus, Kocuria genus, Lachnoanaerobaculum genus, Lactobacillus genus, Lactococcus genus, Lautropia genus, Legionella genus, Leptotrichia genus, Listeria genus, Megasphaera genus, Methanobrevibacter genus, Microbacterium genus, Micrococcus genus, Mitsuokella genus, Mobiluncus genus, Mogibacterium genus, Moraxella genus, Morganella genus, Mycobacterium genus, Mycoplasma genus, Neisseria genus, Nocardia genus, Olsenella genus, Oribacterium genus, Paracoccus genus, Parascardovia genus, Parvimonas genus, Peptoniphilus genus, Peptostreptococcus genus, Porphyromonas genus, Prevotella genus, Propionibacterium (Cutibacterium) genus, Proteus genus, Providencia genus, Pseudomonas genus, Pseudopropionibacterium genus, Pseudoramibacter genus, Pyramidobacter genus, Ralstonia genus, Rothia genus, Scardovia genus, Schlegelella genus, Sebaldella genus, Selenomonas genus, Serratia genus, Simonsiella genus, Slackia genus, Sneathia genus, Solobacterium genus, Staphylococcus genus, Stenotrophomonas genus, Stomatobaculum genus, Streptococcus genus, Tannerella genus, Treponema genus, Ureaplasma genus, Veillonella genus, and Fretibacterium genus.

10. The method according to claim 8, wherein the rRNA genes are rRNA genes of at least two species of bacteria selected from the group consisting of Abiotrophia defectiva, Achromobacter xylosoxidans, Acinetobacter baumannii, Acinetobacter calcoaceticus, Acinetobacter johnsonii, Acinetobacter junii, Acinetobacter lwoffii, Actinomyces dentalis, Actinomyces georgiae, Actinomyces gerencseriae, Actinomyces graevenitzii, Actinomyces israelii, Actinomyces johnsonii, Actinomyces meyeri, Actinomyces naeslundii, Actinomyces odontolyticus, Actinomyces oris, Actinomyces turicensis, Actinomyces viscosus, Aggregatibacter actinomycetemcomitans, Aggregatibacter aphrophilus, Alloprevotella rava, Alloprevotella tannerae, Alloscardovia omnicolens, Anaeroglobus geminatus, Atopobium parvulum, Bacillus cereus, Bacteroides fragilis, Bifidobacterium adolescentis, Bifidobacterium bifidum, Bifidobacterium breve, Bifidobacterium dentium, Bifidobacterium longum, Bordetella pertussis, Brucella abortus, Burkholderia cepacia, Campylobacter concisus, Campylobacter gracilis, Campylobacter rectus, Campylobacter showae, Capnocytophaga gingivalis, Capnocytophaga ochracea, Capnocytophaga sputigena, Cardiobacterium hominis, Catonella morbi, Chlamydia psittaci, Chlamydia trachomatis, Chlamydia pneumoniae, Chryseobacterium indologenes, Citrobacter freundii, Clostridium perfringens, Collinsella aerofaciens, Corynebacterium diphtheriae, Corynebacterium durum, Corynebacterium matruchotii, Corynebacterium pseudodiphtheriticum, Corynebacterium pseudotuberculosis, Corynebacterium renale, Corynebacterium striatum, Corynebacterium xerosis, Coxiella burnetii, Cryptobacterium curtum, Curvibacter delicatus, Dialister invisus, Dialister micraerophilus, Dialister pneumosintes, Eggerthella lenta, Eikenella corrodens, Elizabethkingia meningoseptica, Enterobacter aerogenes, Enterobacter cloacae, Enterococcus avium, Enterococcus faecalis, Enterococcus faecium, Escherichia coli, Eubacterium brachy, Eubacterium infirmum, Eubacterium limosum, Eubacterium minutum, Eubacterium nodatum, Eubacterium saphenum, Eubacterium sulci, Filifactor alocis, Fretibacterium fastidiosum, Fusobacterium necrophorum, Fusobacterium nucleatum, Fusobacterium nucleatum subsp. animalis, Fusobacterium nucleatum subsp. nucleatum, Fusobacterium nucleatum subsp. polymorphum, Fusobacterium nucleatum subsp. vincentii, Fusobacterium periodonticum, Fusobacterium simiae Gemella haemolysans, Gemella morbillorum, Gemella sanguinis, Granulicatella adiacens, Granulicatella balaenopterae, Granulicatella elegans, Haemophilus ducreyi, Haemophilus haemolyticus, Haemophilus influenzae, Haemophilus parainfluenzae, Helicobacter pylori, Kingella denitrificans, Kingella kingae, Kingella oralis, Klebsiella oxytoca, Klebsiella pneumoniae, Lachnoanaerobaculum orale, Lachnoanaerobaculum saburreum, Lactobacillus acidophilus, Lactobacillus brevis, Lactobacillus buchneri, Lactobacillus casei, Lactobacillus crispatus, Lactobacillus delbrueckii, Lactobacillus fermentum, Lactobacillus gasseri, Lactobacillus paracasei, Lactobacillus plantarum, Lactobacillus reuteri, Lactobacillus rhamnosus, Lactobacillus salivarius, Lautropia mirabilis, Legionella pneumophila, Leptotrichia buccalis, Leptotrichia sp. OT 215, Leptotrichia sp. OT 417, Leptotrichia sp. OT 462, Listeria monocytogenes, Megasphaera micronuciformis, Micrococcus luteus, Mogibacterium diversum, Mogibacterium neglectum, Mogibacterium pumilum, Mogibacterium timidum, Mogibacterium vescum, Moraxella catarrhalis, Moraxella lacunata, Morganella morganii, Mycobacterium abscessus, Mycobacterium avium, Mycobacterium chelonae, Mycobacterium fortuitum, Mycobacterium gordonae, Mycobacterium haemophilum, Mycobacterium intracellulare, Mycobacterium kansasii, Mycobacterium marinum, Mycobacterium szulgai, Mycobacterium tuberculosis, Mycobacterium xenopi, Mycoplasma genitalium, Mycoplasma hominis, Mycoplasma orale, Mycoplasma pneumoniae, Mycoplasma salivarium, Neisseria elongata, Neisseria flava, Neisseria flavescens, Neisseria gonorrhoeae, Neisseria meningitidis, Neisseria mucosa, Neisseria sicca, Neisseria subflava, Nocardia asteroides, Nocardia brasilliensis, Nocardia farcinica, Nocardia nova, Olsenella uli, Oribacterium asaccharolyticum, Oribacterium parvum, Parascardovia denticolens, Parvimonas micra, Peptostreptococcus stomatis, Porphyromonas catoniae, Porphyromonas endodontalis, Porphyromonas gingivalis, Porphyromonas pasteri, Prevotella dentalis, Prevotella denticola, Prevotella histicola, Prevotella intermedia, Prevotella loescheii, Prevotella melaninogenica, Prevotella nigrescens, Prevotella oralis, Prevotella oris, Prevotella pallens, Prevotella salivae, Prevotella shahii, Prevotella veroralis, Propionibacterium acnes (Cutibacterium acnes), Propionibacterium granulosum (Cutibacterium granulosum), Propionibacterium propionicus (Pseudopropionibacterium propionicum), Proteus mirabilis, Proteus vulgaris, Providencia stuartii, Pseudomonas aeruginosa, Pseudomonas fluorescens, Pseudomonas putida, Pseudoramibacter alactolyticus, Ralstonia pickettii, Rothia aeria, Rothia dentocariosa, Rothia mucilaginosa, Scardovia inopinata, Schlegelella aquatica, Sebaldella termitidis, Selenomonas flueggei, Selenomonas noxia, Selenomonas sp. OT 478, Selenomonas sputigena, Serratia marcescens, Simonsiella muelleri, Slackia exigua, Solobacterium moorei, SR1 sp. OT 345, Staphylococcus aureus, Staphylococcus epidermidis, Staphylococcus saccharolyticus, Stenotrophomonas maltophilia, Streptococcus agalactiae, Streptococcus anginosus, Streptococcus australis, Streptococcus constellatus, Streptococcus cristatus, Streptococcus dentisani, Streptococcus gordonii, Streptococcus infantis, Streptococcus intermedius, Streptococcus milleri, Streptococcus mitis, Streptococcus mutans, Streptococcus oralis, Streptococcus parasanguinis, Streptococcus pneumoniae, Streptococcus pyogenes, Streptococcus salivarius, Streptococcus sanguinis, Streptococcus sobrinus, Streptococcus tigurinus, Streptococcus vestibularis, Tannerella forsythia, Treponema denticola, Treponema lecithinolyticum, Treponema medium, Treponema pallidum, Treponema socranskii, Treponema vincentii, Ureaplasma urealyticum, Veillonella atypica, Veillonella dispar, Veillonella parvula, and Veillonella rogosae.

11. The method according to claim 1, wherein (c) comprises:
(c1) creating a calibration curve of relationships, using a part or all of the target nucleic acids and the specific nucleic acid group and the control nucleic acid, between amounts before amplification and indicators of amounts after amplification; and
(c2) calculating a total amount of the all target nucleic acids and the specific nucleic acid group in the sample by applying an indicator of a total amount of the amplified all target nucleic acids and the specific nucleic acid group from the sample and the indicator of the amount of the amplified control nucleic acid to the calibration curve created in (c1).

12. The method according to claim 11, wherein the target nucleic acids used in (c1) are a part of the target nucleic acid acids.

13. The method according to claim 11, wherein the calibration curve represents a relationship between
a ratio of an indicator of a total amount of a part or all of the target nucleic acids and the specific nucleic acid group to an indicator of an amount of the control nucleic acid after amplification and
a ratio of a total amount of a part or all of the target nucleic acids and the specific nucleic acid group to an amount of the control nucleic acid before amplification.

14. The method according to claim 1, wherein, in (c) and (d), the indicator of the amount of each amplified target nucleic acid, the indicator of the total amount of the amplified all target nucleic acids and the amplified specific nucleic acid group, and the indicator of the amount of the amplified control nucleic acid are signal intensities obtained by hybridization with probes (x) to (z), respectively, loaded on a DNA chip:
(x) a probe hybridizable respectively with amplified products of two or more types of target nucleic acids;
(y) a probe hybridizable in common with both amplified products of the all target nucleic acids and the specific nucleic acid group in the sample; and
(z) a probe hybridizable with amplified products of the control nucleic acid.

15. The method according to claim 14, wherein (d) comprises:
(d1) hybridizing two or more types of respective target nucleic acids (each target nucleic acid) with the DNA chip and calculating a relational expression on signal intensities between the probe (x) and the probe (y) hybridizable with each target nucleic acid;
(d2) converting the signal intensity of the probe (x) obtained by hybridizing products amplified from the sample with the DNA chip to a value equivalent to the signal intensity of the probe (y) hybridizable with each target nucleic acid using the relational expression calculated in (d1);
(d3) calculating a ratio of the value equivalent to the signal intensity of the probe (y) calculated in (d2) to the signal intensity of the probe (y) obtained by hybridizing products amplified from the sample with the DNA chip; and
(d4) calculating an absolute amount of each target nucleic acid in the sample by multiplying the total amount of the all nucleic acids and the specific nucleic acid group in the sample calculated in (c) by the occupancy calculated in (d3).

16. The method according to claim 14, wherein (b) comprises enriching nucleic acids after amplification for a single chain hybridizable with probes loaded on the DNA chip.

17. The method according to claim 16, wherein the enriching for a single chain is carried out by asymmetric PCR or λ exonuclease treatment.

18. The method according to claim 14, wherein the probe (x) is any of sequences:
(I) a sequence selected from the group consisting of the base sequences as set forth in SEQ ID NO: 3 to 191;
(II) a complementary sequence to the sequence (I); or
(III) a sequence substantially identical with the sequence (I) or (II).

19. The method according to claim 14, wherein the DNA chip is a fiber-type DNA chip.

20. A kit comprising:
one or two pairs of primer sets for co-amplifying all target nucleic acids and a specific nucleic acid group in a sample and a control nucleic acid by PCR method;
the control nucleic acid; and
a DNA chip comprising probes (x) to (z):
(x) a probe hybridizable respectively with amplified products of two or more types of target nucleic acids;
(y) a probe hybridizable in common with both amplified products of the all target nucleic acids and the specific nucleic acid group in the sample; and
(z) a probe hybridizable with amplified products of the control nucleic acid.

21. The kit according to claim 20, wherein the probe (x) is any of sequences:
(I) a sequence selected from the group consisting of the base sequences as set forth in SEQ ID NO: 3 to 191;
(II) a complementary sequence to the sequence (I); or
(III) a sequence substantially identical with the sequence (I) or (II).

22. A DNA chip, comprising probes (x) to (z):
(x) a probe hybridizable respectively with amplified products of two or more types of target nucleic acids;
(y) a probe hybridizable in common with both amplified products of all target nucleic acids and a specific nucleic acid group in a sample; and
(z) a probe hybridizable with amplified products of a control nucleic acid,
wherein the probe (x) is any of sequences:
(IV) a sequence selected from the group consisting of the base sequences as set forth in SEQ ID NO: 43 to 191;
(V) a complementary sequence to the sequence (IV); or
(VI) a sequence substantially identical with the sequence (IV) or (V).


SEQUENCE LISTING (199 sequences)

SEQ ID NO: 1 - DNA, 30 bp, Artificial
  Synthetic DNA
ctattcgaccagcgatatcactacgtaggc

SEQ ID NO: 2 - DNA, 23 bp, Artificial
  Synthetic DNA
cgtattaccgcggctgctggcac

SEQ ID NO: 3 - DNA, 27 bp, Artificial
  Synthetic DNA
gtcaatttggcatgctattaacacacc

SEQ ID NO: 4 - DNA, 27 bp, Artificial
  Synthetic DNA
gtcaagttggcatgctattaacacacc

SEQ ID NO: 5 - DNA, 20 bp, Artificial
  Synthetic DNA
gtcataattctttcccaaga

SEQ ID NO: 6 - DNA, 20 bp, Artificial
  Synthetic DNA
tacacgtacaccttattctt

SEQ ID NO: 7 - DNA, 20 bp, Artificial
  Synthetic DNA
tacattccgaaaaacgtcat

SEQ ID NO: 8 - DNA, 20 bp, Artificial
  Synthetic DNA
ttcaatgcaatactcgtatc

SEQ ID NO: 9 - DNA, 22 bp, Artificial
  Synthetic DNA
cgaagggtaaatgcaaaggggc

SEQ ID NO: 10 - DNA, 22 bp, Artificial
  Synthetic DNA
cgaagggtaaatgcaaaaaggc

SEQ ID NO: 11 - DNA, 20 bp, Artificial
  Synthetic DNA
ctttattcccacataaaagc

SEQ ID NO: 12 - DNA, 18 bp, Artificial
  Synthetic DNA
ttcgcattaggcacgttc

SEQ ID NO: 13 - DNA, 18 bp, Artificial
  Synthetic DNA
aagtaccgtcactgtgtg

SEQ ID NO: 14 - DNA, 19 bp, Artificial
  Synthetic DNA
cacccgttcttctcttaca

SEQ ID NO: 15 - DNA, 21 bp, Artificial
  Synthetic DNA
acagtatgaactttccattct

SEQ ID NO: 16 - DNA, 25 bp, Artificial
  Synthetic DNA
cacgtatctcattttattcccctgt

SEQ ID NO: 17 - DNA, 25 bp, Artificial
  Synthetic DNA
cctcttcttcttattcttcatctgc

SEQ ID NO: 18 - DNA, 17 bp, Artificial
  Synthetic DNA
tccttctaactgttcgc

SEQ ID NO: 19 - DNA, 17 bp, Artificial
  Synthetic DNA
aagtcagcccgtaccca

SEQ ID NO: 20 - DNA, 17 bp, Artificial
  Synthetic DNA
ccacccacaaggagcag

SEQ ID NO: 21 - DNA, 20 bp, Artificial
  Synthetic DNA
gtcaatttggcatgctatta

SEQ ID NO: 22 - DNA, 18 bp, Artificial
  Synthetic DNA
cccaagcagttctatggt

SEQ ID NO: 23 - DNA, 18 bp, Artificial
  Synthetic DNA
gccttcgcaataggtatt

SEQ ID NO: 24 - DNA, 20 bp, Artificial
  Synthetic DNA
caatgggtattcttcttgat

SEQ ID NO: 25 - DNA, 19 bp, Artificial
  Synthetic DNA
caaccattcaagaccaaca

SEQ ID NO: 26 - DNA, 19 bp, Artificial
  Synthetic DNA
tcaaaggcagttgcttagt

SEQ ID NO: 27 - DNA, 19 bp, Artificial
  Synthetic DNA
ctctagctatccagttcag

SEQ ID NO: 28 - DNA, 20 bp, Artificial
  Synthetic DNA
tttctttcttcccaactgaa

SEQ ID NO: 29 - DNA, 19 bp, Artificial
  Synthetic DNA
ccagtactctagttacaca

SEQ ID NO: 30 - DNA, 20 bp, Artificial
  Synthetic DNA
tagttatacagtttccaacg

SEQ ID NO: 31 - DNA, 19 bp, Artificial
  Synthetic DNA
tatgcagtttccaacgcaa

SEQ ID NO: 32 - DNA, 17 bp, Artificial
  Synthetic DNA
gccgtgacttgctggtt

SEQ ID NO: 33 - DNA, 17 bp, Artificial
  Synthetic DNA
ctgtcctcttctgcact

SEQ ID NO: 34 - DNA, 19 bp, Artificial
  Synthetic DNA
gggtaaatgcaaaaaggca

SEQ ID NO: 35 - DNA, 18 bp, Artificial
  Synthetic DNA
ttagccgtccctttctgg

SEQ ID NO: 36 - DNA, 19 bp, Artificial
  Synthetic DNA
tcccctcttgcactcaagt

SEQ ID NO: 37 - DNA, 18 bp, Artificial
  Synthetic DNA
tctcccctcttgcactca

SEQ ID NO: 38 - DNA, 18 bp, Artificial
  Synthetic DNA
cacacgttcttgacttac

SEQ ID NO: 39 - DNA, 18 bp, Artificial
  Synthetic DNA
ccgtcactgtgtaagctt

SEQ ID NO: 40 - DNA, 20 bp, Artificial
  Synthetic DNA
cacgtatctcattttattcc

SEQ ID NO: 41 - DNA, 21 bp, Artificial
  Synthetic DNA
cctcttcttcttattcttcat

SEQ ID NO: 42 - DNA, 18 bp, Artificial
  Synthetic DNA
tattcgcaagaaggcctt

SEQ ID NO: 43 - DNA, 20 bp, Artificial
  Synthetic DNA
aaaaagcagtgccttgttcc

SEQ ID NO: 44 - DNA, 20 bp, Artificial
  Synthetic DNA
gcgcttcataacccggctac

SEQ ID NO: 45 - DNA, 17 bp, Artificial
  Synthetic DNA
aacccacccacaaacga

SEQ ID NO: 46 - DNA, 21 bp, Artificial
  Synthetic DNA
ttcccaactaaaagcagttta

SEQ ID NO: 47 - DNA, 22 bp, Artificial
  Synthetic DNA
tcttaacaaaggtaccgtcacc

SEQ ID NO: 48 - DNA, 22 bp, Artificial
  Synthetic DNA
cctacgcttacttaaccaccta

SEQ ID NO: 49 - DNA, 20 bp, Artificial
  Synthetic DNA
ccctaggacagaggcttaca

SEQ ID NO: 50 - DNA, 20 bp, Artificial
  Synthetic DNA
aaaccctgcgcttaaggtgc

SEQ ID NO: 51 - DNA, 22 bp, Artificial
  Synthetic DNA
cccctactacagagttttacga

SEQ ID NO: 52 - DNA, 20 bp, Artificial
  Synthetic DNA
ccgtctctactgtatatagt

SEQ ID NO: 53 - DNA, 20 bp, Artificial
  Synthetic DNA
gtcaaggcgctaacagttac

SEQ ID NO: 54 - DNA, 20 bp, Artificial
  Synthetic DNA
agttaacgtcaatcacctag

SEQ ID NO: 55 - DNA, 20 bp, Artificial
  Synthetic DNA
gcgggatagtcagtcaggtg

SEQ ID NO: 56 - DNA, 19 bp, Artificial
  Synthetic DNA
cagtttccgatgcagttcc

SEQ ID NO: 57 - DNA, 20 bp, Artificial
  Synthetic DNA
cgtgcagttccgtccacctc

SEQ ID NO: 58 - DNA, 20 bp, Artificial
  Synthetic DNA
taaccacaagattattcgtc

SEQ ID NO: 59 - DNA, 21 bp, Artificial
  Synthetic DNA
agctgtcgatattagcaacag

SEQ ID NO: 60 - DNA, 20 bp, Artificial
  Synthetic DNA
gtgcttaatgaggttaagcc

SEQ ID NO: 61 - DNA, 20 bp, Artificial
  Synthetic DNA
accaccgacttgaaggacca

SEQ ID NO: 62 - DNA, 22 bp, Artificial
  Synthetic DNA
ggtacattcactatggtacacg

SEQ ID NO: 63 - DNA, 22 bp, Artificial
  Synthetic DNA
tacatgcatctcagctacacgt

SEQ ID NO: 64 - DNA, 20 bp, Artificial
  Synthetic DNA
acacgtgactcttgttattc

SEQ ID NO: 65 - DNA, 20 bp, Artificial
  Synthetic DNA
agtcagacgttgggcgccta

SEQ ID NO: 66 - DNA, 20 bp, Artificial
  Synthetic DNA
cacgtgactgactttatccc

SEQ ID NO: 67 - DNA, 20 bp, Artificial
  Synthetic DNA
cctactttcagcgcactcaa

SEQ ID NO: 68 - DNA, 20 bp, Artificial
  Synthetic DNA
aatagggacacgtccctaac

SEQ ID NO: 69 - DNA, 22 bp, Artificial
  Synthetic DNA
cacgtgcatcaaattattctcg

SEQ ID NO: 70 - DNA, 20 bp, Artificial
  Synthetic DNA
acgtgggctcttttatcccc

SEQ ID NO: 71 - DNA, 20 bp, Artificial
  Synthetic DNA
acacgtgattgactttatcc

SEQ ID NO: 72 - DNA, 20 bp, Artificial
  Synthetic DNA
acccactgcaaaaccagggt

SEQ ID NO: 73 - DNA, 20 bp, Artificial
  Synthetic DNA
tctcttcttccctgctaaca

SEQ ID NO: 74 - DNA, 20 bp, Artificial
  Synthetic DNA
actattcgcactaggcacgt

SEQ ID NO: 75 - DNA, 22 bp, Artificial
  Synthetic DNA
gtaccgtcacccaaactcaata

SEQ ID NO: 76 - DNA, 20 bp, Artificial
  Synthetic DNA
ccaacaatttaaccacttac

SEQ ID NO: 77 - DNA, 20 bp, Artificial
  Synthetic DNA
cgtcattcgtcttctgccaa

SEQ ID NO: 78 - DNA, 18 bp, Artificial
  Synthetic DNA
ctggtaagttaccgtcac

SEQ ID NO: 79 - DNA, 20 bp, Artificial
  Synthetic DNA
cacactcgttcttgacttac

SEQ ID NO: 80 - DNA, 20 bp, Artificial
  Synthetic DNA
tacacacgttcttcccctac

SEQ ID NO: 81 - DNA, 21 bp, Artificial
  Synthetic DNA
gtcgattaccgtcatcagatg

SEQ ID NO: 82 - DNA, 20 bp, Artificial
  Synthetic DNA
ttcctccaaaacttattcct

SEQ ID NO: 83 - DNA, 21 bp, Artificial
  Synthetic DNA
cgtcaaatcctcgcactattc

SEQ ID NO: 84 - DNA, 22 bp, Artificial
  Synthetic DNA
accgtcaattcctctaactatt

SEQ ID NO: 85 - DNA, 20 bp, Artificial
  Synthetic DNA
cagtttcgcgaggtattaac

SEQ ID NO: 86 - DNA, 20 bp, Artificial
  Synthetic DNA
caccagacgaagtccgtgct

SEQ ID NO: 87 - DNA, 22 bp, Artificial
  Synthetic DNA
ctaggtccggtagcaagctacc

SEQ ID NO: 88 - DNA, 20 bp, Artificial
  Synthetic DNA
taccttcccccgctcgactt

SEQ ID NO: 89 - DNA, 23 bp, Artificial
  Synthetic DNA
atcagtagcaagctacctctctc

SEQ ID NO: 90 - DNA, 22 bp, Artificial
  Synthetic DNA
cgctaagatcagtagcaagcta

SEQ ID NO: 91 - DNA, 21 bp, Artificial
  Synthetic DNA
gtattaatctcggtagcctcc

SEQ ID NO: 92 - DNA, 24 bp, Artificial
  Synthetic DNA
ctaggttaggtagcaagctacatt

SEQ ID NO: 93 - DNA, 22 bp, Artificial
  Synthetic DNA
caccaaaacaccacaaaagtga

SEQ ID NO: 94 - DNA, 20 bp, Artificial
  Synthetic DNA
actaacccaccccgcaaaaa

SEQ ID NO: 95 - DNA, 20 bp, Artificial
  Synthetic DNA
tccagtattagcacccattt

SEQ ID NO: 96 - DNA, 22 bp, Artificial
  Synthetic DNA
atgcgaagatcagtgaatatcc

SEQ ID NO: 97 - DNA, 20 bp, Artificial
  Synthetic DNA
aagttggagcatcatcgttc

SEQ ID NO: 98 - DNA, 20 bp, Artificial
  Synthetic DNA
gccaatgagtgaatatccag

SEQ ID NO: 99 - DNA, 21 bp, Artificial
  Synthetic DNA
ggaagtctcgaagtattcggt

SEQ ID NO: 100 - DNA, 22 bp, Artificial
  Synthetic DNA
gagctttctcgagtcttccatg

SEQ ID NO: 101 - DNA, 22 bp, Artificial
  Synthetic DNA
cggaatcattatgctatcgggt

SEQ ID NO: 102 - DNA, 20 bp, Artificial
  Synthetic DNA
caagctttcttcctgatgcc

SEQ ID NO: 103 - DNA, 22 bp, Artificial
  Synthetic DNA
tagagccaaggatttctttccg

SEQ ID NO: 104 - DNA, 20 bp, Artificial
  Synthetic DNA
actacaggacatgttccgat

SEQ ID NO: 105 - DNA, 21 bp, Artificial
  Synthetic DNA
ctctcaagatcccgaaagatc

SEQ ID NO: 106 - DNA, 20 bp, Artificial
  Synthetic DNA
gtacgccgctctcaagatcc

SEQ ID NO: 107 - DNA, 20 bp, Artificial
  Synthetic DNA
taaccacaacgccttcctcc

SEQ ID NO: 108 - DNA, 20 bp, Artificial
  Synthetic DNA
gtcacccaaggagcaagctc

SEQ ID NO: 109 - DNA, 22 bp, Artificial
  Synthetic DNA
ctgacactaaacagtggtccta

SEQ ID NO: 110 - DNA, 20 bp, Artificial
  Synthetic DNA
tcgagtacccgcagcaagct

SEQ ID NO: 111 - DNA, 20 bp, Artificial
  Synthetic DNA
cggacagtagtgcaagcact

SEQ ID NO: 112 - DNA, 20 bp, Artificial
  Synthetic DNA
aataacgcgaggtcttgcga

SEQ ID NO: 113 - DNA, 20 bp, Artificial
  Synthetic DNA
ttaaccttaacgccttcctc

SEQ ID NO: 114 - DNA, 22 bp, Artificial
  Synthetic DNA
cctcgctgaaagtactttacaa

SEQ ID NO: 115 - DNA, 20 bp, Artificial
  Synthetic DNA
cggttattaaccacaacacc

SEQ ID NO: 116 - DNA, 20 bp, Artificial
  Synthetic DNA
agctctctgtgctaccgttc

SEQ ID NO: 117 - DNA, 20 bp, Artificial
  Synthetic DNA
tctctgtgctaccgttcgac

SEQ ID NO: 118 - DNA, 20 bp, Artificial
  Synthetic DNA
tctttggtcttgcgacgtta

SEQ ID NO: 119 - DNA, 20 bp, Artificial
  Synthetic DNA
ctctcaggtgcggctatgca

SEQ ID NO: 120 - DNA, 20 bp, Artificial
  Synthetic DNA
aggtccccctctttgtgctt

SEQ ID NO: 121 - DNA, 20 bp, Artificial
  Synthetic DNA
cgtcaatgagcaaaggtatt

SEQ ID NO: 122 - DNA, 20 bp, Artificial
  Synthetic DNA
gacatacgtaattaccgcgg

SEQ ID NO: 123 - DNA, 20 bp, Artificial
  Synthetic DNA
gcttgcgacatacgtaatta

SEQ ID NO: 124 - DNA, 20 bp, Artificial
  Synthetic DNA
atgttgtcccggtctaagag

SEQ ID NO: 125 - DNA, 20 bp, Artificial
  Synthetic DNA
cttcaaggcatattcctacg

SEQ ID NO: 126 - DNA, 20 bp, Artificial
  Synthetic DNA
ttacaaccctcaggccttct

SEQ ID NO: 127 - DNA, 21 bp, Artificial
  Synthetic DNA
ttacaaccctcaggccttctt

SEQ ID NO: 128 - DNA, 22 bp, Artificial
  Synthetic DNA
cctcttcaaggcatattcctac

SEQ ID NO: 129 - DNA, 20 bp, Artificial
  Synthetic DNA
cagtattatctgaccgtccc

SEQ ID NO: 130 - DNA, 22 bp, Artificial
  Synthetic DNA
aggctaatcttaaagcgccagg

SEQ ID NO: 131 - DNA, 21 bp, Artificial
  Synthetic DNA
cagctttcgtcctcagacatt

SEQ ID NO: 132 - DNA, 20 bp, Artificial
  Synthetic DNA
gcttcctaacttcgttcgac

SEQ ID NO: 133 - DNA, 20 bp, Artificial
  Synthetic DNA
gtcagggcttatgggtatta

SEQ ID NO: 134 - DNA, 21 bp, Artificial
  Synthetic DNA
acatctgactcaatcaaccgc

SEQ ID NO: 135 - DNA, 22 bp, Artificial
  Synthetic DNA
tattaaccttgacaccttcctc

SEQ ID NO: 136 - DNA, 20 bp, Artificial
  Synthetic DNA
cagaagacatgcgtcttgag

SEQ ID NO: 137 - DNA, 22 bp, Artificial
  Synthetic DNA
gagtacctccgaagaggccttt

SEQ ID NO: 138 - DNA, 20 bp, Artificial
  Synthetic DNA
attgcccacgtgttactcac

SEQ ID NO: 139 - DNA, 20 bp, Artificial
  Synthetic DNA
cttcttctccacctaccgtc

SEQ ID NO: 140 - DNA, 20 bp, Artificial
  Synthetic DNA
ccactcgagtgtctccgaag

SEQ ID NO: 141 - DNA, 20 bp, Artificial
  Synthetic DNA
aggcttatcccggtgtgcag

SEQ ID NO: 142 - DNA, 21 bp, Artificial
  Synthetic DNA
acccaacagcatgcactgaaa

SEQ ID NO: 143 - DNA, 20 bp, Artificial
  Synthetic DNA
gtcagttacttcccagagac

SEQ ID NO: 144 - DNA, 22 bp, Artificial
  Synthetic DNA
ccgcttggttttgctcagaatt

SEQ ID NO: 145 - DNA, 22 bp, Artificial
  Synthetic DNA
actcaatactgagcaaaccctc

SEQ ID NO: 146 - DNA, 22 bp, Artificial
  Synthetic DNA
gaggacctcgttggaatacata

SEQ ID NO: 147 - DNA, 22 bp, Artificial
  Synthetic DNA
ctgcttgaaggtaggttggata

SEQ ID NO: 148 - DNA, 21 bp, Artificial
  Synthetic DNA
ccctaactttatccccataca

SEQ ID NO: 149 - DNA, 20 bp, Artificial
  Synthetic DNA
gtatggtacctgcaaatagg

SEQ ID NO: 150 - DNA, 21 bp, Artificial
  Synthetic DNA
ctttaatcgtcgttggatgcc

SEQ ID NO: 151 - DNA, 20 bp, Artificial
  Synthetic DNA
agacgatgccatgaggtatt

SEQ ID NO: 152 - DNA, 20 bp, Artificial
  Synthetic DNA
tctttacccattaccgtcac

SEQ ID NO: 153 - DNA, 20 bp, Artificial
  Synthetic DNA
cccaagattacacttccgac

SEQ ID NO: 154 - DNA, 20 bp, Artificial
  Synthetic DNA
gattaacgcctgcaccctcc

SEQ ID NO: 155 - DNA, 20 bp, Artificial
  Synthetic DNA
cgcctgcaccctccgtatta

SEQ ID NO: 156 - DNA, 20 bp, Artificial
  Synthetic DNA
taccgctcgattgcatgtgt

SEQ ID NO: 157 - DNA, 20 bp, Artificial
  Synthetic DNA
aactttatcaccttcctccc

SEQ ID NO: 158 - DNA, 20 bp, Artificial
  Synthetic DNA
gcggttcaatatattatccg

SEQ ID NO: 159 - DNA, 20 bp, Artificial
  Synthetic DNA
ataagtgacagcaaaaccgt

SEQ ID NO: 160 - DNA, 20 bp, Artificial
  Synthetic DNA
ttagccagctggatttcttt

SEQ ID NO: 161 - DNA, 20 bp, Artificial
  Synthetic DNA
cgacagagtagattccgatg

SEQ ID NO: 162 - DNA, 24 bp, Artificial
  Synthetic DNA
agtctagtgtaaacaccaaacctc

SEQ ID NO: 163 - DNA, 21 bp, Artificial
  Synthetic DNA
actcctaccaacgttcttctc

SEQ ID NO: 164 - DNA, 21 bp, Artificial
  Synthetic DNA
catctactagcgatgcaattg

SEQ ID NO: 165 - DNA, 20 bp, Artificial
  Synthetic DNA
ttaagtaccgtcacagcatg

SEQ ID NO: 166 - DNA, 20 bp, Artificial
  Synthetic DNA
acctttcatccagtctcgat

SEQ ID NO: 167 - DNA, 20 bp, Artificial
  Synthetic DNA
cgtcaaatcctcgcactatt

SEQ ID NO: 168 - DNA, 20 bp, Artificial
  Synthetic DNA
cacacctgactgactatccc

SEQ ID NO: 169 - DNA, 21 bp, Artificial
  Synthetic DNA
ccactaatcagcactctagca

SEQ ID NO: 170 - DNA, 20 bp, Artificial
  Synthetic DNA
aaactaagaggcacatgacc

SEQ ID NO: 171 - DNA, 21 bp, Artificial
  Synthetic DNA
gttttaaatgctgacttgggg

SEQ ID NO: 172 - DNA, 20 bp, Artificial
  Synthetic DNA
gcataaactcttcctcaacc

SEQ ID NO: 173 - DNA, 20 bp, Artificial
  Synthetic DNA
aaccgttatccccaagttga

SEQ ID NO: 174 - DNA, 20 bp, Artificial
  Synthetic DNA
tctcagtcccgcactttcat

SEQ ID NO: 175 - DNA, 20 bp, Artificial
  Synthetic DNA
tattaacctcatcgccttcc

SEQ ID NO: 176 - DNA, 20 bp, Artificial
  Synthetic DNA
gtaacgtcaatcgatgaggt

SEQ ID NO: 177 - DNA, 21 bp, Artificial
  Synthetic DNA
aaatggtacagtcaaactcta

SEQ ID NO: 178 - DNA, 20 bp, Artificial
  Synthetic DNA
catgcgaaccaaagttctta

SEQ ID NO: 179 - DNA, 20 bp, Artificial
  Synthetic DNA
ccggacgttatcccccacta

SEQ ID NO: 180 - DNA, 20 bp, Artificial
  Synthetic DNA
gctgaatccaggagcaagct

SEQ ID NO: 181 - DNA, 21 bp, Artificial
  Synthetic DNA
gcgagtaacgtcaattgatga

SEQ ID NO: 182 - DNA, 20 bp, Artificial
  Synthetic DNA
cccctctacgagactctagc

SEQ ID NO: 183 - DNA, 22 bp, Artificial
  Synthetic DNA
gtattaagctcaccaccttcct

SEQ ID NO: 184 - DNA, 20 bp, Artificial
  Synthetic DNA
ctaacatcagagaagcaagc

SEQ ID NO: 185 - DNA, 20 bp, Artificial
  Synthetic DNA
aagaccgtctttcacttttg

SEQ ID NO: 186 - DNA, 20 bp, Artificial
  Synthetic DNA
cttacacatatgttcttccc

SEQ ID NO: 187 - DNA, 20 bp, Artificial
  Synthetic DNA
atgcaacatccactcttatg

SEQ ID NO: 188 - DNA, 20 bp, Artificial
  Synthetic DNA
caagtgcaccttttaagcaa

SEQ ID NO: 189 - DNA, 22 bp, Artificial
  Synthetic DNA
catgcgttagtctctcttatgc

SEQ ID NO: 190 - DNA, 20 bp, Artificial
  Synthetic DNA
ccatctcatagtggagcaat

SEQ ID NO: 191 - DNA, 21 bp, Artificial
  Synthetic DNA
cccaccatcattcttctctaa

SEQ ID NO: 192 - DNA, 15 bp, Artificial
  Synthetic DNA
tacgggaggcagcag

SEQ ID NO: 193 - DNA, 19 bp, Artificial
  Synthetic DNA
crgggtatctaatccygtt

SEQ ID NO: 194 - DNA, 25 bp, Artificial
  Synthetic DNA
gagaagcctacacaaacgtaacgtc

SEQ ID NO: 195 - DNA, 23 bp, Artificial
  Synthetic DNA
ctctaaagaccgctctatctcgg

SEQ ID NO: 196 - DNA, 458 bp, Artificial
  Synthetic DNA
gtgagaagcctacacaaacgtaacgtcagggctaagacaaacgctaacggtacaccctagatgggagcttgtagctagatcgctaagtcctaccgacatgtaggcatactcacgaaggcaattccctgaaagcctcgtcttatcccgaacttggcatctgctgatacgtcaggttgaacgcgtacatttacctgtcatgcgtgggccttctccgaatagcctacgtagtgatatcgctggtcgaataggcggattgctcataaatgcacattggctaaggcccacggaacacgaatcacgtgagatcacttactattcgacggaactactatacgcaccgggacatgcaagtagcgtcccacaagcataaggaactctatactcgccatctacgcagctacaggggatacacgtatgagcggttacgaagtaaagccgagatagagcggtctttagag

SEQ ID NO: 197 - DNA, 18 bp, Artificial
  Synthetic DNA
  misc_feature (5)..(5): n is inosine
taccngggtatctaatcc

SEQ ID NO: 198 - DNA, 458 bp, Artificial
  Synthetic DNA
gttcctacgggaggcagcagtagggctaagacaaacgctaacggtacaccctagatgggagcttgtagctagatcgctaagtcctaccgacatgtaggcatactcacgaaggcaattccctgaaagcctcgtcttatcccgaacttggcatctgctgatacgtcaggttgaacgcgtacatttacctgtcatgcgtgggccttctccgaatagcctacgtagtgatatcgctggtcgaataggcggattgctcataaatgcacattggctaaggcccacggaacacgaatcacgtgagatcacttactattcgacggaactactatacgcaccgggacatgcaagtagcgtcccacaagcataaggaactctatactcgccatctacgcagctacaggggatacacgtatgagcggttacgaagtaaagggtagcaaacaggattagataccctgaaa

SEQ ID NO: 199 - DNA, 463 bp, Artificial
  Synthetic DNA
gttcctacgggaggcagcagtagggctaagacaaacgctaacggtacaccctagatgggagcttgtagctagatcgctaagtcctaccgacatgtaggcatactcacgaaggcaattccctgaaagcctcgtcttatcccgaacttggcatctgctgatacgtcaggttgaacgcgtacatttacctgtcatgcgtgggccttctccgaatagcctacgtagtgatatcgctggtcgaataggcggattgctcataaatgcacattggctaaggcccacggaacacgaatcacgtgagatcacttactattcgacggaactactatacgcaccgggacatgcaagtagcgtcccacaagcataaggaactctatactcgccatctacgcagctacaggggatacacgtatgagcggttacgaagtaaagggtagcaacaggattagataccctggtagtccac